(19) United States
(12) Patent Application Publication
(10) Pub. No.: US 2013/0203715 A1
(43) Pub. Date: Aug. 8, 2013

(54) USE OF TRP CHANNEL AGONISTS TO TREAT INFECTIONS

(71) Applicants: David L. Hava, Natick, MA (US); Robert W. Clarke, Medfield, MA (US)

(72) Inventors: David L. Hava, Natick, MA (US); Robert W. Clarke, Medfield, MA (US)

(73) Assignee: Pulmatrix, Inc., Lexington, MA (US)

(21) Appl. No.: 13/810,762

(22) PCT Filed: Jul. 20, 2011
(86) PCT No.: PCT/US11/44628

§ 371 Date: Mar. 13, 2013

Related U.S. Application Data
(60) Provisional application No. 61/365,840, filed on Jul. 20, 2010.

(51) Int. Cl. A61K 31/496; A61K 31/23; A61K 31/495; A61K 31/196; A61K 31/045; A61K 31/05; A61K 31/513; A61K 45/06; A61K 31/357

(52) U.S. Cl. CPC A61K 31/496; A61K 45/06; A61K 31/23; A61K 31/495; A61K 31/357; A61K 31/045; A61K 31/05; A61K 31/513; A61K 31/196
USPC 514/171; 514/510; 514/252.13; 514/255.04; 514/450; 514/729; 514/731; 514/274; 514/567

(57) ABSTRACT

Methods are described for treating or preventing a respiratory infection by administering an effective amount of a TRP channel agonist.

### RELATED APPLICATIONS

This application claims the benefit of U.S. Provisional Application No. 61/365,840, filed on Jul. 20, 2010. The entire contents of the foregoing application is hereby incorporated by reference.

### RELATED APPLICATIONS

Transient receptor potential (TRP) channels represent a large superfamily of homologous membrane proteins. TRP channels are expressed throughout the body, and several TRP channel family members can be expressed on a single cell type. TRP channels are composed of six-transmembrane (6TM) polypeptide subunits that combine to form tetramers. These tetramers form pores in the membrane that are permeable to cations (e.g., Ca²⁺ and Na⁺). TRP channel activation allows for rapid, yet controlled, entry of one or more cations into a cell, and are involved in sensory transduction in response to a diverse array of physiological stimuli. (Clapham D E. TRP channels as cellular sensors. Nature. 2003. 426:517-524.) TRP channels are classified into subfamilies based on sequence homology, which include the TRPC, TRPV, TRPM and TRPA1 subfamilies.

TRPC (where “C” represents “classic” or “canonical”) channel subfamily members are G-protein-coupled receptor (GPCR) or receptor tyrosine kinase activated channels. TRPC1, TRPC4 (CCE2), and TRPC5 (CCE1) are highly homologous, are expressed in the central nervous system (CNS) and form homo or heteromeric channels. (Clapham 2003) TRPC3, TRPC6, and TRPC7 are also highly expressed in smooth and cardiac muscle cells and may be involved in the regulation of vascular tone, airway resistance, and/or cardiac function. (See Clapham 2003; Trebak M, Vazquez G, Bird G S, Putney, J W. The TRPC3/6/7 subfamily of cation channels. Cell Calcium. 2003. 33(5-6):451-461.)

The TRPV (where “V” represents “vanilloid”) channel subfamily members are more selective for Ca²⁺ than other TRP subfamilies, and TRPV5 and TRPV6 are the most Ca²⁺ selective TRP channels. (Clapham 2003) TRPV1 (VR1), TRPV2 (VRL1, OTRPC2), TRPV3 (VRL2) and TRPV4 (OTRPC4, VR-OAC) are activated by elevated temperatures.

These four TRPV channels are also thought to be activated by cell stretching that is likely due to detecting changes in extracellular tonicity, and specifically hypotonicity. (Birder L A, et al. Altered urinary bladder function in mice lacking the vanilloid receptor TRPV 1. Nat Neurosci. 2002. 5(9):856-860; Iwata Y, et al. A novel mechanism of myocyte degeneration involving the Ca2+-permeable growth factor-regulated channel. J Cell Biol. 2003. 161(5):957-967; Hu H Z, et al. 2-aminoethoxydiphenyl borate is a common activator of TRPV1, TRPV2, and TRPV3. J Biol Chem. 2004. 279(34):35741-35748.) TRPV5 (ECaC1, CaT2) and TRPV6 (ECaC2, CaT1) are the only TRPV channels not known to possess thermosensory activity. Both TRPV5 and TRPV6 are expressed in the intestines, are constitutively active, and are inhibited by intracellular Ca²⁺ concentrations, which suggests a role in calcium absorption. (Clapham D E. SnapShot: Mammalian TRP Channels. Cell. 2007. 129(1):220; den Dekker E, et al. The epithelial calcium channels, TRPV5 & TRPV6: from identification towards regulation. Cell Calcium. 2003. 33(5-6):497-507; Hoenderop J G, et al. Homo- and heterotetrameric architecture of the epithelial Ca2+ channels TRPV5 and TRPV6. EMBO J. 2003. 22(4):776-785.)

The TRPM (where “M” represents “melastatin”) channel subfamily takes its name from the observed over-expression of TRPM1 (MLSN) transcripts in certain metastatic melanomas. TRPM2 (hTRPC7, LTRPC2) is gated by binding of ADP-ribose and NAD (nicotinic adenine dinucleotide) to its C-terminal hydrolase domain. TRPM3 (MLSN2, LTRPC3), like TRPV4, is sensitive to hypotonicity, but there is little homology to suggest a common mechanism of action. TRPM4 (LTRPC4, MLS2s, CAN[4b]) and TRPM5 (Mtr1, LTRPC5) are the only TRP channels that are monovalent cation-selective. TRPM4 is widely expressed, and TRPM4 deficient mice have enhanced anaphylactic responses. (Clapham 2003; Clapham 2007) TRPM5 is also widely expressed, and plays a role in the taste perception of sweet, bitter and umami (amino acid) sensations. (Zhang Y, et al. Coding of sweet, bitter, and umami tastes: different receptor cells sharing similar signaling pathways. Cell. 2003. 112(3):293-301.) TRPM6 (CHAK2) and TRPM7 (CHAK, TRP-PLIK, LTRPC7) contain a functional kinase domain, but this domain is not necessary for the channel activity. (Runnels L W, et al. The TRPM7 channel is inactivated by PIP(2) hydrolysis. Nat Cell Biol. 2002. 4(5):329-336.; Schmitz C, et al. Regulation of vertebrate cellular Mg2+ homeostasis by TRPM7. Cell. 2003. 114(2):191-200.) TRPM7 is thought to play a role in monitoring intracellular energy stores by sensing Mg-ATP levels. (Nadler M J, et al. LTRPC7 is a Mg-ATP regulated divalent cation channel required for cell viability. Nature. 2001. 411:590-595.) TRPM8 (Trp-p8, CMR1) is involved in detecting “cooling” and noxious cold sensations from ˜8° C. to 28° C. Menthol and icilin are agonists that enhance the sensory transduction of TRPM8. (Clapham 2003; McKemy D D, et al. Identification of a cold receptor reveals a general role for TRP channels in thermosensation. Nature. 2002. 416:52-58; Peier A M, et al. A TRP channel that senses cold stimuli and menthol. Cell. 2002. 108(5):705-715.)

TRPA1 (ANKTM1, P120), (where “A” represents “ankyrin”) is the only member of the TRPA subfamily. TRPA1 is activated by temperatures below 15° C. Although there is no significant homology to TRPM8, TRPA1 is activated by the TRPM8 agonist icilin. (Clapham 2007; Story G M, et al. ANKTM1, a TRP-like channel expressed in nociceptive neurons, is activated by cold temperatures. Cell. 2003. 112(6):819-829.) TRPA1 is usually co-expressed in TRPV1 positive dorsal root ganglion neurons that do not express TRPM8. (Clapham 2003; Kobayashi K, et al. Distinct expression of TRPM8, TRPA1, and TRPV1 mRNAs in rat primary afferent neurons with adelta/c-fibers and colocalization with trk receptors. J Comp Neurol. 2005. 493(4):596-606.)

Some TRP channel agonists, such as the vanilloid capsaicin, are known pain relievers. (Tominaga M, Julius D. Capsaicin receptor in the pain pathway. Jpn J. Pharmacol. 2000. 83(1):20-24; Cortright D N, Szallasi A. TRP channels and pain. Curr Pharm Des. 2009. 15(15):1736-1749.)

Certain TRPV3 agonists may be useful for treating inflammatory-associated conditions, including asthma and inflammatory bowel disorder (See, e.g., WIPO Patent Publication WO2008065666) or allergic and non-allergic rhinitis (See US Patent Publication No. 20090286811).

Certain TRPV1 agonists might be useful to treat rhinitis and herpes (See U.S. Pat. No. 7,632,519).

Current therapies for respiratory tract infections involve the administration of anti-viral agents, anti-bacterial agents, or anti-fungal agents for the treatment, prevention, or amelioration of viral, bacterial, and fungal respiratory tract infections, respectively. Unfortunately, in some cases, there are no therapies available, infections are refractory to therapies, or the occurrence of side effects outweighs the benefits of the administration of a therapeutic agent. The use of anti-viral or anti-bacterial agents for treatment of viral or bacterial tract infections may produce side effects or result in the emergence of resistant strains. The administration of anti-fungal agents may cause renal failure or bone marrow dysfunction and may not be effective against fungal infection in patients with suppressed immune systems. Additionally, the infection-causing organisms (e.g., a virus, a bacterium, or a fungus) may be resistant or develop resistance to the administered therapeutic agent or combination of therapeutic agents. In fact, microorganisms that develop resistance to administered therapeutic agents often develop pleiotropic drug or multidrug resistance, that is, resistance to therapeutic agents that act by mechanisms different from the mechanisms of how the administered agents act. Thus, as a result of drug resistance, many infections prove refractory to a wide array of standard treatment protocols. Therefore, new therapies for the treatment, prevention, management, and/or amelioration of respiratory tract infections and symptoms thereof are needed.

### SUMMARY

The invention relates to a method of treating or preventing infections. The method includes administering to an individual an effective amount of a TRP channel agonist selected from the group consisting of TRPV2, TRPV3, TRPV4, TRPC6, TRPM6, TRPA1, and combinations thereof.

In one aspect, the invention is a method of treating or preventing a respiratory infection, comprising administering to an individual an effective amount of a TRP channel agonist selected from the group consisting of TRPV2, TRPV3, TRPV4, TRPC6, TRPM6, TRPA1, and combinations thereof. In a particular aspect, an agonist of TRPV4 is administered.

In one aspect, the invention relates to a method of treating a respiratory infection, comprising administering to an individual having a respiratory infection an effective amount of a TRP channel agonist selected from the group consisting of Allyl isothiocyanate (AITC), Benyzl isothiocyanate (BITC), Phenyl isothiocyanate, Isopropyl isothiocyanate, methyl isothiocyanate, diallyl disulfide, acrolein (2-propenal), disulfuram (Antabuse®), farnesyl thiosalicylic acid (FTS), farnesyl thioacetic acid (FTA), chlodantoin (Sporostacin®, topical fungicidal), (15-d-PGJ2), 5,8,11,14 eicosatetraynoic acid (ETYA), dibenzoazepine, mefenamic acid, fluribiprofen, keoprofen, diclofenac, indomethacin, SC alkyne (SCA), pentenal, mustard oil alkyne (MOA), iodoacetamine, iodoacetamide alkyne, (2-aminoethyl) methanethiosulphonate (MTSEA), 4-hydroxy-2-noneal (HNE),4-hydroxy xexenal (HHE), 2-chlorobenzalmalononitrile, N-chloro tosylamide (chloramine-T), formaldehyde, isoflurane, isovelleral, hydrogen peroxide, URB597, thiosulfinate, Allicin (a specific thiosulfinate), flufenamic acid, niflumic acid, carvacrol, eugenol, menthol, gingerol, icilin, methyl salicylate, arachidonic acid, cinnemaldehyde, super sinnemaldehyde, tetrahydrocannabinol (THC or Δ⁹-THC), cannabidiol (CBD), cannabichromene (CBC), cannabigerol (CBG), THC acid (THC-A), CBD acid (CBD-A), Compound 1 (AMG5445), 4-methyl-N-[2,2,2-trichloro-1-(4-chlorophenylsulfanyl)ethyl]benzamide, N-[2,2,2-trichloro-1-(4-chlorophenylsulfanyl) ethyl]acetamid, AMG9090, AMG5445, 1-oleoyl-2-acetyl-sn-glycerol (OAG), carbachol, diacylglycerol (DAG), 1,2-Didecanoylglycerol, flufenamate/flufenamic acid, niflumate/niflumic acid, hyperforin, 2-aminoethoxydiphenyl borate (2-APB), diphenylborinic anhydride (DPBA), delta-9-tetrahydrocannabinol (Δ⁹-THC or THC), cannabiniol (CBN), 2-APB, O-1821, 11-hydroxy-Δ⁹-tetrahydrocannabinol, nabilone, CP55940, HU-210, HU-211/dexanabinol, HU-331, HU-308, JWH-015, WINS 5,212-2, 2-Arachidonoylglycerol (2-AG), Arvil, PEA, AM404, O-1918, JWH-133, incensole, incensole acetate, menthol, eugenol, dihydrocarveol, carveol, thymol, vanillin, ethyl vanillin, cinnemaldehyde, 2 aminoethoxydiphenyl borate (2-APB), diphenylamine (DPA), diphenylborinic anhydride (DPBA), camphor, (+)-borneol, (−)-isopinocampheol, (−)-fenchone, (−)-trans-pinocarveol, isoborneol, (+)-camphorquinone, (−)-α-thujone, α-pinene oxide, 1,8-cineole/eucalyptol, 6-tert-butyl-m-cresol, carvacrol, p-sylenol, kreosol, propofol, p-cymene, (−)-isoppulegol, (−)-carvone, (+)-dihydrocarvone, (−)-menthone, (+)-linalool, geraniol, 1-isopropyl-4-methyl-bicyclo[3.1.0]hexan-4-ol, 4αPDD, GSK1016790A, 5′6′Epoxyeicosatrienoic (5′6′-EET), 8′9′Epoxyeicosatrienoic (8′9′-EET), APP44-1, RN1747, Formulation Ib WO200602909, Formulation IIb WO200602909, Formulation IIc WO200602929, Formulation IId WO200602929, Formulation IIIb WO200602929, Formulation IIIc WO200602929, arachidonic acid (AA), 12-O-Tetradecanoylphorbol-13-acetate (TPA)/phorbol 12-myristate 13-acetate (PMA), bisandrographalide (BAA), incensole, incensole acetate, Compound IX WO2010015965, Compound X WO2010015965, Compound XI WO2010015965, Compound XII WO2010015965, WO2009004071, WO2006038070, WO2008065666, Formula VII WO2010015965, Formula IV WO2010015965, dibenzoazepine, dibenzooxazepine, Formula I WO2009071631, N-{(1S)-1-[({(4R)-1-[(4-chlorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(4-fluorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(2-cyanophenyl)sulfonyl]-3-oxohexahydro-1H-azepin-4-yl}amino)carbonyl]-3-methylbutyl}-1-methyl-1H-indole-2-carboxamide, and N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide.

In another aspect, the invention relates to a method of treating a respiratory infection, comprising administering to an individual having a respiratory infection an effective amount of a TRP channel agonist selected from the group consisting of Allyl isothiocyanate (AITC), Benyzl isothiocyanate (BITC), Phenyl isothiocyanate, Isopropyl isothiocyanate, methyl isothiocyanate, diallyl disulfide, acrolein (2-propenal), disulfuram (Antabuse®), farnesyl thiosalicylic acid (FTS), farnesyl thioacetic acid (FTA), chlodantoin (Sporostacin®, topical fungicidal), (15-d-PGJ2), 5,8,11,14 eicosatetraynoic acid (ETYA), dibenzoazepine (WO9747611), dibenzoxazepine (WO9747611), dibenz[b,f]-[1,4]oxazepine (CR), 11H-dibenz[b,e]azepine, 1,2 naphthoquione, 1,3-dihydroxynaphthalene, 2 methyl-1,4-naphthoquinone, 1-nitronaphthalene, hydroquinone, 4-phenyl-1,2-dihydronaphthalene, 3,5-ditert-butylphenol, 2,4-ditert-butylphenol, 1,3 butadiene, [(3E)-1-phenyl-1,3-pentadienyl]benzene, [(2Z)-3-phenyl-2-butenyl]benzene, mefenamic acid, fluribiprofen, keoprofen, diclofenac, indomethacin, SC alkyne (SCA), pentenal, mustard oil alkyne (MOA), iodoacetamine, iodoacetamide alkyne, (2-aminoethyl) methanethiosulphonate (MTSEA), 4-hydroxy-2-noneal (HNE), 4-hydroxy xexenal (HHE), 2-chlorobenzalmalononitrile, N-chloro tosylamide (chloramine-T), aldehyde, acetaldehyde (US2009269280), formaldehyde, O-anisaldehyde, isoflurane, isovelleral, hydrogen peroxide, URB597, thiosulfinate, Allicin (a specific thiosulfinate), flufenamic acid, niflumic acid, carvacrol, eugenol, menthol, gingerol, propofol, thymol, 2-tert-butyl-5-methylphenol, icilin, methyl salicylate, arachidonic acid, cinnemaldehyde, super cinnemaldehyde, 10-hydroxy-2-decenoic acid, 10-hydroxydecanoic acid, 4-oxo-2-nonenal (4-ONE), 1-chloroacetophenone (CN), bromobenzyl cyanide, Compounds CA3, 13-19 and 21-27 from Defalco J et al., Bioorg Med Chem Lett. 2010 20(1):276-279, 2-chlorobenzylidene malononitrile (CS), morphanthridine, Compounds 6 and 32 from Gijsen H J et al., J Med Chem 2010, 53(19):7011-7020, methyl vinyl ketone, mesityl oxide, acrylic acid N-hydroxysuccinimide ester, hydrocinnamic acid N-hydroxysuccinimide ester, 3-(2-Pyridyldithio)propionic acid N-hydroxysuccinimide ester, N-acetyl-p-benzo-quinoneimine, 1′-acetoxychavicol acetate, piperine, isopiperine, isochavicine, piperanine, piperolein A, piperolein B, (2E,4E)-N-Isobutyl-2,4-decadienamide, nitro-oleic acid (OA-NO₂), 2-chloroacetophenone, styrene, naphthalene, indolinone compounds (US2011009379), tetrahydrocannabinol (THC or Δ⁹-THC), cannabidiol (CBD), cannabichromene (CBC), cannabinol (CBN), cannabigerol (CBG), THC acid (THC-A), tetrahydrocannabivarin (THCVA), CBD acid (CBD-A), Compound 1 (AMG5445), 4-methyl-N-[2,2,2-trichloro-1-(4-chlorophenylsulfanyl)ethyl]benzamide, N-[2,2,2-trichloro-1-(4-chlorophenylsulfanyl)ethyl]acetamid, AMG9090, AMG5445, 1-oleoyl-2-acetyl-sn-glycerol (OAG), carbachol, diacylglycerol (DAG), 1,2-Didecanoylglycerol, flufenamate/flufenamic acid, niflumate/niflumic acid, hyperforin, 2-aminoethoxydiphenyl borate (2-APB), diphenylborinic anhydride (DPBA), delta-9-tetrahydrocannabinol (Δ⁹-THC or THC), cannabiniol (CBN), 2-APB, O-1821, 11-hydroxy-Δ⁹-tetrahydrocannabinol, nabilone, CP55940, HU-210, HU-211/dexanabinol, HU-331, HU-308, JWH-015, WINS 5,212-2, 2-Arachidonoylglycerol (2-AG), Arvil, PEA, AM404, O-1918, JWH-133, incensole, incensole acetate, menthol, eugenol, dihydrocarveol, carveol, thymol, vanillin, ethyl vanillin, cinnemaldehyde, 2 aminoethoxydiphenyl borate (2-APB), diphenylamine (DPA), diphenylborinic anhydride (DPBA), camphor, (+)-borkkkol, (−)-isopinocampheol, (−)-fenchone, (−)-trans-pinocarveol, isoborneol, (+)-camphorquinone, (−)-α-thujone, α-pinene oxide, 1,8-cineole/eucalyptol, 6-tert-butyl-m-cresol, carvacrol, p-sylenol, kreosol, propofol, p-cymene, (−)-isoppulegol, (−)-carvone, (+)-dihydrocarvone, (−)-menthone, (+)-linalool, geraniol, farnesyl pyrophosphate, farnesyl diphosphate, isopentenyl pyrophosphate, 1-isopropyl-4-methyl-bicyclo[3.1.0]hexan-4-ol, 4αPDD, GSK1016790A, 5′6′Epoxyeicosatrienoic (5′6′-EET), 8′9′Epoxyeicosatrienoic (8′9′-EET), APP44-1, RN1747, Formulation Ib WO200602909, Formulation IIb WO200602909, Formulation IIc WO200602929, Formulation IId WO200602929, Formulation IIIb WO200602929, Formulation IIIc WO200602929, arachidonic acid (AA), 12-O-T etradecanoylphorbol-13-acetate (TPA)/phorbol 12-myristate 13-acetate (PMA), bisandrographalide (BAA), incensole, incensole acetate, Compound IX WO2010015965, Compound X WO2010015965, Compound XI WO2010015965, Compound XII WO2010015965, WO2009004071, WO2006038070, WO2008065666, Formula VII WO2010015965, Formula IV WO2010015965, dibenzoazepine, dibenzooxazepine, Formula I WO2009071631, N-{(1S)-1-[({(4R)-1-[(4-chlorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(4-fluorophenyl)sulfonyl]-3-oxohexahydro-1H-azepin-4-yl}amino)carbonyl]-3-methylbutyl}-1-benzothiophen-2-carboxamide, N-{(1S)-1[({(4R)-1-[(2-cyanophenyl)sulfonyl]-3-oxohexahydro-1H-azepin-4-yl}amino)carbonyl]-3-methylbutyl}-1-methyl-1H-indole-2-carboxamide, N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide, N-(4-Hydroxyphenyl)-5Z,8Z,11Z,14Z-eicosatetraenamide (AM404) and anandamide.

The respiratory infection can be a bacterial infection. For example, the bacterial infection can be caused by Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp., Yersinia enterocolitica, Mycobacterium tuberculosis, Bordetella pertussis, Bordetella bronchiseptica, Brucella spp., Brucella abortus, Brucella melitensis, Brucella suis, Chlamydophila psittaci, Clostridium tetani, Streptococcus pyogenes, Corynebacterium diphtheriae, Neisseria meningitides, Enterococcus faecalis, Francisella tularensis, Bacillus anthracis, Helicobacter pylori, Leptospira spp., Leptospira interrogans, Listeria monocytogenes, Rickettsia rickettsii, Salmonella spp., Shigella sonnei, Vibrio cholerae, or Yersinia pestis.

The respiratory infection can be a viral infection. For example, the viral infection can be caused by influenza virus, rhinovirus, parainfluenza virus, respiratory syncytial virus (RSV), metapneumovirus, adenovirus, herpes simplex virus, cytomegalovirus (CMV), coronavirus, hantavirus, coxsackievirus, rhinovirus, enterovirus, or human bocavirus (HBoV).

In some embodiments, the TRP channel agonists is administered as an aerosol to the respiratory tract of the individual.

In one embodiment, the method may further comprise administering one or more co-therapeutic agents selected from the group consisting of mucoactive or mucolytic agents, surfactants, cough suppressants, expectorants, steroids, bronchodilators, antihistamines, anti-inflammatory agents, antibiotics, and antiviral agents.

The invention also relates to a method of treating a respiratory infection, comprising administering to an individual having a respiratory infection, an effective amount of 4αPDD.

The invention also relates to a method of treating a respiratory infection, comprising administering to an individual having a respiratory infection, an effective amount of GSK1016790A.

The invention also relates to a method of treating a respiratory infection, comprising administering to an individual having a respiratory infection, an effective amount of RN1747.

### BRIEF DESCRIPTION OF THE DRAWINGS

FIG. 1 shows graphical illustrations of the effects of two broad-spectrum TRP channel antagonists on the ability of an 8× calcium-sodium (Ca:Na) formulation to reduce influenza (Influenza A/Panama/2007/99) infection of Calu-3 cells. Viral infection was measured by quantifying viral titer using a 50% Tissue Culture Infectious Dose (TCID₅₀) assay. FIG. 1A is a graph illustrating that ruthenium red (RR) abrogates the ability of an 8×Ca:Na formulation to reduce influenza infection. FIG. 1B is a graph illustrating that SKF96365 attenuates the ability of an 8×Ca:Na formulation to reduce influenza infection.

FIG. 2A is a graph illustrating that RR attenuates the ability of an 8×Ca:Na formulation to reduce parainfluenza (hPIV3) infection of Calu-3 cells.

FIG. 2B is a graph illustrating that RR attenuates the ability of an 8×Ca:Na formulation to reduce rhinovirus (Rv⁶) infection of Calu-3 cells.

FIG. 3 is a graph illustrating that RR attenuates the ability of an 8×Ca:Na formulation to reduce influenza infection of normal human bronchial epithelial (NHBE) cells.

FIG. 4A is a graph illustrating that RR attenuates the ability of a dry powder, Formulation I (10% leucine, 58.6% calcium lactate, 31.4% sodium chloride, weight percent), to reduce influenza infection of Calu-3 cells.

FIG. 4B is a graph illustrating that RR attenuates the ability of a dry powder, Formulation II (10% leucine, 39.6% calcium chloride, 50.4% sodium sulfate, weight percent), to reduce influenza infection of Calu-3 cells.

FIG. 5 shows a graphical illustration of the expression levels of various TRP channels in Calu-3 and NHBE cells as assayed by quantitative PCR (qPCR). Cycle threshold (Ct) values for the genes were calculated as the expression level of the genes relative to the expression level of a housekeeping gene (GAPDH).

FIG. 6 is a graph illustrating that the Transient Receptor Potential V4 (TRPV4) antagonist RN1734, in a dose-dependent manner, abrogates the ability of an 8×Ca:Na formulation to reduce influenza infection of Calu-3 cells.

FIG. 7 shows graphical illustrations of the effects of three TRPV4 agonists on influenza infection of Calu-3 cells. FIG. 7A is a graph illustrating that 4αPDD reduces influenza infection. FIG. 7B is a graph illustrating that GSK1016790A reduces influenza infection in a dose-dependent manner. FIG. 7C is a graph illustrating that RN1747 reduces influenza infection in a dose-dependent manner.

FIG. 8A is a graph illustrating that 4αPDD reduces parainfluenza infection of Calu-3 cells.

FIG. 8B is a graph illustrating that 4αPDD reduces rhinovirus infection of Calu-3 cells.

FIG. 9 is a graph illustrating that 4αPDD and GSK1016790A reduce influenza infection of NHBE cells.

FIG. 10 shows graphical illustrations of the effects of multiple-target TRP channel agonists on influenza infection of Calu-3 cells relative to the effect of 4αPDD. FIG. 10A is a graph illustrating that menthol, an agonist of TRPM8 and TRPV3, reduces influenza infection. FIG. 10B is a graph illustrating that carvacrol, an agonist of TRPV3 and TRPA1, reduces influenza infection. FIG. 10C is a graph illustrating that icilin, an agonist of TRPM8 and TRPA1, modestly reduces influenza infection.

FIG. 11A is a graph illustrating that flufenamic acid, an agonist of TRPC6 and TRPA1, reduces influenza infection of Calu-3 cells.

FIG. 11B is a graph illustrating that allicin, an agonist of TRPA1, reduces influenza infection of Calu-3 cells.

### DETAILED DESCRIPTION

As described and exemplified herein, the inventors conducted studies into the anti-infective properties of certain calcium-containing formulations, and surprisingly discovered that TRP channels are involved in the anti-infective effect. The invention provides methods for treating or preventing infections of mucosal surfaces comprising administering an effective amount of a TRP channel agonist to an individual in need thereof. The TRP channel agonist can be administered to the individual by any suitable route of administration, depending on the mucosal surface to be treated. For example, mucosal surfaces include a mucosal surface of the alimentary tract, respiratory tract, urogenital tract, eye, eustation tube, and the like, such as buccal mucosa, esophageal mucosa, gastric mucosa, intestinal mucosa, nasal mucosa, olfactory mucosa, oral mucosa, bronchial mucosa, uterine mucosa, endometrium, urethra and penile mucosa.

The inventors further discovered, that agonists of TRP channels surprisingly have anti-infective activity in models of respiratory tract infections. Methods of treating and preventing respiratory tract infections are described and exemplified herein.

As used herein, the term “respiratory infection” refers to an infection of the respiratory tract that is caused by a microbial pathogen. Common symptoms of a respiratory infection include fever, cough, shortness of breath (dyspnea), and/or wheezing. Clinically, respiratory infections can be diagnosed, for example, by culturing the infecting organism, by clinical exam, or other suitable methods, such as chest x-ray. The diagnosis of a respiratory infection does not require that the presence of an infective pathogen in the respiratory tract of the individual be confirmed.

The term “respiratory tract” as used herein includes the upper respiratory tract (e.g., nasal passages, nasal cavity, throat, pharynx), respiratory airways (e.g., larynx, trachea, bronchi, bronchioles) and lungs (e.g., respiratory bronchioles, alveolar ducts, alveolar sacs, alveoli).

The term “aerosol” as used herein refers to any preparation of a fine mist of particles (including liquid and non-liquid particles, e.g., dry powders), typically with a volume median geometric diameter of about 0.1 to about 30 microns or a mass median aerodynamic diameter of between about 0.5 and about 10 microns. Preferably the volume median geometric diameter for the aerosol particles is less than about 10 microns. The preferred volume median geometric diameter for aerosol particles is about 5 microns. For example, the aerosol can contain particles that have a volume median geometric diameter between about 0.1 and about 30 microns, between about 0.5 and about 20 microns, between about 0.5 and about 10 microns, between about 1.0 and about 3.0 microns, between about 1.0 and 5.0 microns, between about 1.0 and 10.0 microns, between about 5.0 and 15.0 microns.

Preferably the mass median aerodynamic diameter is between about 0.5 and about 10 microns, between about 1.0 and about 3.0 microns, or between about 1.0 and 5.0 microns.

To facilitate the preparation of a clear and concise specification, the invention is further described with reference to preferred embodiments of treating respiratory infections. It is intended and to be understood that the methods can be used to treat infections of any mucosal tissue.

### Methods of Treating Respiratory Infections

In one aspect, the invention provides methods for treating or preventing a respiratory infection that comprises administering an effective amount of a TRP channel agonist to an individual in need thereof. The TRP channel agonist can be administered to the individual by any suitable route of administration, such as orally, parenterally, by inhalation or other suitable route. Preferably, the TRP channel agonist is administered by inhalation.

The individual to be treated in accordance with the invention may have a diagnosed respiratory infection, such as an infection diagnosed by clinical exam, by diagnostic procedure (e.g., chest x-ray) and/or confirmed presence of an infective agent in the respiratory tract of the subject (e.g., using a suitable microbiological or molecular diagnostic test). The subject to be treated in accordance with the invention may be at risk for a respiratory infection. Generally, such subjects are exposed to infectious agents more frequently than the general population, or are more susceptible to infection than the general population. Individuals at risk for a respiratory infection include, for example, health care workers, individuals with chronic lung diseases (e.g., asthma, chronic bronchitis, emphysema, chronic obstructive pulmonary disease, cystic fibrosis, bronchiectasis), individuals who are immunosuppressed, infants, newborns and young (e.g., humans younger than about 12 years of age), and elderly (e.g., humans older that about 65 or 70 years of age).

Accordingly, in some embodiments, the invention is a method for the treatment or prevention of respiratory infection in an individual with a chronic underlying respiratory disease, such as asthma, chronic bronchitis, emphysema, chronic obstructive pulmonary disease, cystic fibrosis, bronchiectasis, comprising administering an effective amount of a TRP channel agonist to the individual.

The TRP channel agonist administered in accordance with the methods of the invention is preferably an agonist of one or more of TRPV2, TRPV3, TRPV4, TRPC6, TRPM6, and TRPA1. One or more TRP channel agonists can be administered. In some embodiments, two or more agonists that activate the same or different TRP channels are administered. Although specific agonists of TRPV2, TRPV3, TRPV4, TRPC6, TRPM6, and TRPA1 may be beneficial in some instances, the invention does not require the use of specific agonists. In some embodiments, the TRP channel agonist administered does not activate TRPV1. In other embodiments, the TRP channel agonist administered does not activate a TRP channel other than a channel selected from the group consisting of TRPV2, TRPV3, TRPV4, TRPC6, TRPM6, TRPA1, and combinations thereof. Preferred TRP channel agonists are non-toxic when administered to the respiratory tract.

Exemplary agonists of TRPA1 that can be administered in accordance with the method of the invention include Allyl isothiocyanate (AITC), Benyzl isothiocyanate (BITC), Phenyl isothiocyanate, Isopropyl isothiocyanate, methyl isothiocyanate, diallyl disulfide, acrolein (2-propenal), disulfuram (Antabuse®), farnesyl thiosalicylic acid (FTS), farnesyl thioacetic acid (FTA), chlodantoin (Sporostacin®, topical fungicidal), 15-d-PGJ2, 5, 8, 11, 14 eicosatetraynoic acid (ETYA), dibenzoazepine (WO9747611), dibenzoxazepine (WO9747611), dibenz[b,f]-[1,4]oxazepine (CR), 11H-dibenz[b,e]azepine, 1,2 naphthoquione, 1,3-dihydroxynaphthalene, 2 methyl-1,4-naphthoquinone, 1-nitronaphthalene, hydroquinone, 4-phenyl-1,2-dihydronaphthalene, 3,5-ditert-butylphenol, 2,4-ditert-butylphenol, 1,3 butadiene, [(3E)-1-phenyl-1,3-pentadienyl]benzene, [(2Z)-3-phenyl-2-butenyl]benzene, mefenamic acid, fluribiprofen, keoprofen, diclofenac, indomethacin, SC alkyne (SCA), pentenal, mustard oil alkyne (MOA), iodoacetamine, iodoacetamide alkyne, (2-aminoethyl)methanethiosulphonate (MTSEA), 4-hydroxy-2-noneal (HNE), 4-hydroxy xexenal (HHE), 2-chlorobenzalmalononitrile, N-chloro tosylamide (chloramine-T), aldehyde, acetaldehyde (US2009269280), formaldehyde, O-anisaldehyde, isoflurane, isovelleral, hydrogen peroxide, URB597, thiosulfinate, Allicin (a specific thiosulfinate), flufenamic acid, niflumic acid, carvacrol, eugenol, menthol, gingerol, propofol, thymol, 2-tert-butyl-5-methylphenol, icilin, methyl salicylate, arachidonic acid, cinnemaldehyde, super cinnemaldehyde, 10-hydroxy-2-decenoic acid, 10-hydroxydecanoic acid, 4-oxo-2-nonenal (4-ONE), 1-chloroacetophenone (CN), bromobenzyl cyanide, Compounds CA3, 13-19 and 21-27 from Defalco J et al., Bioorg Med Chem Lett. 2010 20(1):276-279, 2-chlorobenzylidene malononitrile (CS), morphanthridine, Compounds 6 and 32 from Gijsen H J et al., J Med Chem 2010, 53(19):7011-7020, methyl vinyl ketone, mesityl oxide, acrylic acid N-hydroxysuccinimide ester, hydrocinnamic acid N-hydroxysuccinimide ester, 3-(2-Pyridyldithio)propionic acid N-hydroxysuccinimide ester, N-acetyl-p-benzo-quinoneimine, 1′-acetoxychavicol acetate, piperine, isopiperine, isochavicine, piperanine, piperolein A, piperolein B, (2E,4E)-N-Isobutyl-2,4-decadienamide, nitro-oleic acid (OA-NO₂), 2-chloroacetophenone, styrene, naphthalene, indolinone compounds (US2011009379), tetrahydrocannabinol (THC or Δ⁹-THC), cannabidiol (CBD), cannabichromene (CBC), cannabinol (CBN), cannabigerol (CBG), THC acid (THC-A), tetrahydrocannabivarin (THCVA), CBD acid (CBD-A), Compound 1 (AMG5445), 4-methyl-N-[2,2,2-trichloro-1-(4-chlorophenylsulfanyl)ethyl]benzamide, N-[2,2,2-trichloro-1-(4-chlorophenylsulfanyl)ethyl]acetamid, AMG9090, AMG5445, and the compounds disclosed in WO 2009/071631 (e.g., a compound of Formula I).

Exemplary agonists of TRPC6 that can be administered in accordance with the method of the invention include 1-oleoyl-2-acetyl-sn-glycerol (OAG), carbachol, diacylglycerol (DAG), 1,2-Didecanoylglycerol, flufenamate/flufenamic acid, niflumate/niflumic acid, hyperforin, and the compounds disclosed in WO 2010/015965 (e.g., a compound of Formula IV, compound IX, compound X, compound XI, compounds XII).

Exemplary agonists of TRPM6 that can be administered in accordance with the method of the invention include 2-aminoethoxydiphenyl borate (2-APB).

Exemplary agonists of TRPV2 that can be administered in accordance with the method of the invention include diphenylborinic anhydride (DPBA), delta-9-tetrahydrocannabinol (Δ⁹-THC or THC), cannabiniol (CBN), cannabidiol (CBP), 2-APB, probenecid, O-1821, 11-hydroxy-Δ⁹-tetrahydrocannabinol, nabilone, CP55940, HU-210, HU-211/dexanabinol, HU-331, HU-308, JWH-015, WINS 5,212-2, 2-Arachidonoylglycerol (2-AG), Arvil, PEA, AM404, O-1918, and JWH-133.

Exemplary agonists of TRPV3 that can be administered in accordance with the method of the invention include Incensole, incensole acetate, a compound disclosed in WO 2008/065666 (e.g., a compound of Formula I or Formula II, compound IA), menthol, eugenol, dihydrocarveol, carveol, thymol, vanillin, ethyl vanillin, cinnemaldehyde, 2 aminoethoxydiphenyl borate (2-APB), diphenylamine (DPA), diphenylborinic anhydride (DPBA), camphor, (+)-borneol, (−)-isopinocampheol, (−)-fenchone, (−)-trans-pinocarveol, isoborneol, (+)-camphorquinone, (−)-α-thujone, α-pinene oxide, 1,8-cineole/eucalyptol, 6-tert-butyl-m-cresol, carvacrol, p-xylenol, kreosol, propofol, p-cymene, (−)-isoppulegol, (−)-carvone, (+)-dihydrocarvone, (−)-menthone, (+)-linalool, geraniol, farnesyl pyrophosphate, farnesyl diphosphate, isopentenyl pyrophosphate, and 1-isopropyl-4-methyl-bicyclo[3.1.0]hexan-4-ol.

Exemplary agonists of TRPV4 that can be administered in accordance with the method of the invention include 4αPDD, GSK1016790A, 5′6′Epoxyeicosatrienoic (5′6′-EET), 8′9′Epoxyeicosatrienoic (8′9′-EET), APP44-1, RN1747, Formulation Ib WO200602909, Formulation IIb WO200602909, Formulation IIc WO200602929, Formulation IId WO200602929, Formulation IIIb WO200602929, Formulation IIIc WO200602929, arachidonic acid (AA), 12-O-Tetradecanoylphorbol-13-acetate (TPA)/phorbol 12-myristate 13-acetate (PMA), bisandrographalide (BAA), and compounds disclosed in WO 2006/029209 (e.g., a compound of Formula I, II, IIa, or III, N-{(1S)-1[({(4R)-1-[(4-chlorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(4-fluorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(2-cyanophenyl)sulfonyl]-3-oxohexahydro-1H-azepin-4-yl}amino)carbonyl]-3-methylbutyl}-1-methyl-1H-indole-2-carboxamide, N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide), N-(4-Hydroxyphenyl)-5Z,8Z,11Z,14Z-eicosatetraenamide (AM404) and anandamide.

The respiratory infection may be caused by a microbial pathogen, such as bacteria or viruses. Exemplary bacterial pathogens that cause respiratory infections that may be treated or prevented by the methods of the invention include, but are not limited to, Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter, Acinetobacter baumannii, methicillin-resistant Staphylococcus aureus, Stenotrophomonas maltophilia, Burkholderia spp., Yersinia enterocolitica, Mycobacterium tuberculosis, Bordetella pertussis, Bordetella bronchiseptica, Brucella spp., Brucella abortus, Brucella melitensis, Brucella suis, Chlamydophila psittaci, Clostridium tetani, Streptococcus pyogenes, Corynebacterium diphtheriae, Neisseria meningitides, Enterococcus faecalis, Francisella tularensis, Bacillus anthracis, Helicobacter pylori, Leptospira spp., Leptospira interrogans, Listeria monocytogenes, Rickettsia rickettsii, Salmonella spp., Shigella sonnei, Vibrio cholerae, Yersinia pestis, and combinations thereof.

Exemplary viral pathogens that cause respiratory infections that may be treated or prevented by the methods of the invention include, but are not limited to, Orthomyxoviridae (e.g., influenza virus A or B), Paramyxoviridae (e.g., respiratory syncytial virus (RSV) and metapneumovirus), Adenoviridae (e.g., adenovirus), Herpesviridae (e.g., herpes simplex virus, cytomegalovirus (CMV), and parainfluenza virus), Coronaviridae (e.g., coronavirus (SARS-CoV)), Bunyaviridae (e.g., hantavirus), Picornaviridae (e.g., coxsackievirus, rhinovirus, and enteroviruses), Parvoviridae (e.g., human bocavirus (HBoV)), rhinovirus and combinations thereof. In particular embodiments, the subject is infected by influenza virus, parainfluenza virus, or rhinovirus. In a more particular embodiment, the subject is infected by influenza virus.

The invention provides a method for treating or preventing a respiratory infection that comprises administering an effective amount of a TRPV2 channel agonist to a subject in need thereof.

The invention provides a method for treating or preventing a respiratory infection that comprises administering an effective amount of a TRPV3 channel agonist to a subject in need thereof.

The invention provides a method for treating or preventing a respiratory infection that comprises administering an effective amount of a TRPV4 channel agonist to a subject in need thereof.

The invention provides a method for treating or preventing a respiratory infection that comprises administering an effective amount of a TRPC6 channel agonist to a subject in need thereof.

The invention provides a method for treating or preventing a respiratory infection that comprises administering an effective amount of a TRPM6 channel agonist to a subject in need thereof.

The invention provides a method for treating or preventing a respiratory infection that comprises administering an effective amount of a TRPA1 channel agonist to a subject in need thereof.

The invention provides a method for the treatment or prevention of respiratory infection in an individual with a chronic underlying respiratory disease, such as asthma, chronic bronchitis, emphysema, chronic obstructive pulmonary disease, cystic fibrosis, bronchiectasis, comprising administering an effective amount of a TRPV2 channel agonist to the individual.

The invention provides a method for the treatment or prevention of respiratory infection in an individual with a chronic underlying respiratory disease, such as asthma, chronic bronchitis, emphysema, chronic obstructive pulmonary disease, cystic fibrosis, bronchiectasis, comprising administering an effective amount of a TRPV3 channel agonist to the individual.

The invention provides a method for the treatment or prevention of respiratory infection in an individual with a chronic underlying respiratory disease, such as asthma, chronic bronchitis, emphysema, chronic obstructive pulmonary disease, cystic fibrosis, bronchiectasis, comprising administering an effective amount of a TRPV4 channel agonist to the individual.

The invention provides a method for the treatment or prevention of respiratory infection in an individual with a chronic underlying respiratory disease, such as asthma, chronic bronchitis, emphysema, chronic obstructive pulmonary disease, cystic fibrosis, bronchiectasis, comprising administering an effective amount of a TRPC6 channel agonist to the individual.

The invention provides a method for the treatment or prevention of respiratory infection in an individual with a chronic underlying respiratory disease, such as asthma, chronic bronchitis, emphysema, chronic obstructive pulmonary disease, cystic fibrosis, bronchiectasis, comprising administering an effective amount of a TRPM6 channel agonist to the individual.

The invention provides a method for the treatment or prevention of respiratory infection in an individual with a chronic underlying respiratory disease, such as asthma, chronic bronchitis, emphysema, chronic obstructive pulmonary disease, cystic fibrosis, bronchiectasis, comprising administering an effective amount of a TRPA1 channel agonist to the individual.

In a specific embodiment, the invention provides a method of treating a respiratory infection, comprising administering to an individual having a respiratory infection an effective amount of an agonist of TRPV4, wherein the agonist is 4αPDD. In another embodiment, the invention provides a method of treating a respiratory infection, comprising administering to an individual having a respiratory infection an effective amount of an agonist of TRPV4, wherein the agonist is GSK1016790A. In a further embodiment, the invention provides a method of treating a respiratory infection, comprising administering to an individual having a respiratory infection an effective amount of an agonist of TRPV4, wherein the agonist is RN 1747.

The invention provides a method for treating or preventing a respiratory infection that comprises administering an effective amount of one or more TRP channel agonists shown in Table 1 to an individual in need thereof.

The invention provides a method for the treatment or prevention of respiratory infection in an individual with a chronic underlying respiratory disease, such as asthma, chronic bronchitis, emphysema, chronic obstructive pulmonary disease, cystic fibrosis, bronchiectasis, comprising administering an effective amount of one or more TRP channel agonists shown in Table 1 to the individual.

In particular embodiments, the methods of the invention comprise administering the effective amount of a TRP channel agonist to the respiratory tract of the individual (e.g., a patient with a respiratory infection). Delivery to the respiratory tract is preferably by inhalation of an aerosol, such as a dry powder aerosol or a nebulized aerosol.

In preferred embodiments of the methods described herein, the individual has or is at risk for a viral respiratory infection.

The methods of the invention can further comprise administering a co-therapeutic agent, such as mucoactive or mucolytic agents, surfactants, cough suppressants, expectorants, steroids such as a corticosteroid, bronchodilators, antihistamines, anti-inflammatory agents, antibiotics, and antivirals. Cotherapeutic agents can be administered in any desired way, provided that there is overlap in the pharmacological activity of the TRP channel agonist and the co-therapeutic agent. For example, the co-therapeutic agent can be administered before, after or substantially concurrently with the TRP channel agonist.

Generally, the TRP channel agonist is administered to an individual as a component of a pharmaceutical composition.

### Modes of Administration

The TRP channel agonist can be administered in any suitable way, such as, parenterally (e.g., intravenous, intramuscular, intraperotineal, or subcutaneous injection), topically, transdermally, via suppository (e.g., rectal or intravaginal administration), orally or by inhalation. The TRP channel agonist can be administered in a single dose or multiple doses as indicated.

Preferably, the TRP channel agonist is administered to the respiratory tract (e.g., to the mucosal surface of the respiratory tract), and can be administered in any suitable form, such as a solution, a suspension, a spray, a mist, a foam, a gel, a vapor, droplets, particles, or a dry powder. In more preferred embodiments, the TRP channel agonist is aerosolized for administration to the respiratory tract. TRP channel agonists can be aerosolized for administration via the oral airways using any suitable method and/or device, and many suitable methods and devices are conventional and well-known in the art. For example, TRP channel agonists can be aerosolized using a metered dose inhaler (e.g., a pressurized metered dose inhaler (pMDI) including HFA propellant, or a non-HFA propellant) with or without a spacer or holding chamber, a nebulizer, an atomizer, a continuous sprayer, an oral spray or a dry powder inhaler (DPI). TRP channel agonists can be aerosolized for administration via the nasal airways using a nasal pump or sprayer, a metered dose inhaler (e.g., a pressurized metered dose inhaler (pMDI) including HFA propellant, or a non-HFA propellant) with or without a spacer or holding chamber, a nebulizer with or without a nasal adapter or prongs, an atomizer, a continuous sprayer, or a DPI. TRP channel agonists can also be delivered to the nasal mucosal surface via, for example, nasal wash and to the oral mucosal surfaces via, for example, an oral wash. TRP channel agonists can be delivered to the mucosal surfaces of the sinuses via, for example, nebulizers with nasal adapters and nasal nebulizers with oscillating or pulsatile airflows.

The geometry of the airways is an important consideration when selecting a suitable method for producing and delivering aerosols of TRP channel agonists to the lungs. The lungs are designed to entrap particles of foreign matter that are breathed in, such as dust. There are three basic mechanisms of deposition: impaction, sedimentation, and Brownian motion (J. M. Padfield. 1987. In: D. Ganderton & T. Jones eds. Drug Delivery to the Respiratory Tract, Ellis Harwood, Chicherster, U.K.). Impaction in the upper airways occurs when particles are unable to stay within the air stream, particularly at airway branches. Impacted particles are adsorbed onto the mucus layer covering bronchial walls and eventually cleared from the lungs by mucociliary action. Impaction mostly occurs with particles over 5 μm in aerodynamic diameter. Smaller particles (those less than about 3 μm in aerodynamic diameter) tend to stay within the air stream and to be advected deep into the lungs. Sedimentation often occurs in the lower respiratory system where airflow is slower. Very small particles (those less than about 0.6 μm) can deposit by Brownian motion. Deposition by Brownian motion is generally undesirable because deposition cannot be targeted to the alveoli (N. Worakul & J. R. Robinson. 2002. In: Polymeric Biomaterials, 2^{nd} Ed. S. Dumitriu ed. Marcel Dekker. New York).

For administration, a suitable method (e.g., nebulization, dry powder inhaler) is selected to produce aerosols with the appropriate particle size for preferential delivery to the desired region of the respiratory tract, such as the deep lung (generally particles between about 0.6 microns and 5 microns in diameter), the upper airway (generally particles of about 3 microns or larger diameter), or the deep lung and the upper airway.

Some respiratory infections begin as infections of the upper respiratory airways. For example, influenza virus typically replicates initially in the upper airways and later in the lung epithelia. Therefore, the TRP channel agonist can be delivered to the upper respiratory airway and/or the lung (e.g., deep lung). Delivery to the upper respiratory airways is advantageous for prophylaxis or to prevent an early infection from spreading.

In the methods of the invention, an “effective amount” of a TRP channel agonist is administered to an individual in need thereof. An effective amount is an amount that is sufficient to achieve the desired therapeutic or prophylactic effect, such as an amount sufficient to reduce respiratory infection, to reduce duration of illness, to reduce pathogen burden, to reduce the number of days that infected individuals experience respiratory infection symptoms and/or to decrease the incidence or rate of respiratory infection.

The clinician of ordinary skill can determine appropriate dosage of TRP channel agonist based on the properties of the particular TRP channel agonist selected and other conventional factors, for example, the individual's age, sensitivity or tolerance to drugs, the particular infection to be treated and the individuals overall well-being, and the treating clinician's sound judgment.

### Pharmaceutical Compositions

Pharmaceutical compositions that contain a TRP channel agonist for use in the methods described herein contain at least one TRP channel agonist as an active ingredient, and a pharmaceutically acceptable carrier or diluent, and can optionally contain additional agents. The pharmaceutical composition can be in any desired form, such as a solution, emulsion, suspension, or a dry powder. Preferred pharmaceutical composition, such as solutions and dry powders, can be aerosolized. The pharmaceutical composition can comprise multiple doses or be a unit dose composition as desired.

The pharmaceutical composition is generally prepared in or comprises a physiologically acceptable carrier or excipient. For pharmaceutical composition in the form of solutions, suspensions or emulsions, any suitable carrier or excipient can be included. Suitable carriers include, for example, aqueous, alcoholic/aqueous, and alcohol solutions, emulsions or suspensions, including water, saline, ethanol/water solution, ethanol solution, buffered media, propellants and the like. For pharmaceutical composition in the form of dry powders, suitable carrier or excipients include, for example, sugars (e.g., lactose, trehalose), sugar alcohols (e.g., mannitol, xylitol, sorbitol), amino acids (e.g., glycine, alanine, leucine, isoleucine), dipalmitoylphosphosphatidylcholine (DPPC), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, phospholipids, alkylated sugars, sodium phosphate, maltodextrin, human serum albumin (e.g., recombinant human serum albumin), biodegradable polymers (e.g., PLGA), dextran, dextrin, and the like. If desired, the pharmaceutical composition can also contain additives, preservatives, or fluid, nutrient or electrolyte replenishers (See, generally, Remington's Pharmaceutical Sciences, 17th Edition, Mack Publishing Co., PA, 1985).

The pharmaceutical composition formulation preferably contains a concentration of TRP channel agonist that permits convenient administration of an effective amount of the TRP channel agonist to the respiratory tract. For example, it is generally desirable that liquid formulations not be so dilute so as to require a large amount of the formulation to be nebulized in order to deliver an effective amount to the respiratory tract of a subject. Long administration periods are disfavored, and generally the formulation should be concentrated enough to permit an effective amount to be administered to the respiratory tract (e.g., by inhalation of aerosolized formulation, such as nebulized liquid or aerosolized dry powder) in no more than about 120 minutes, no more than about 90 minutes, no more than about 60 minutes, no more than about 45 minutes, no more than about 30 minutes, no more than about 25 minutes, no more than about 20 minutes, no more than about 15 minutes, no more than about 10 minutes, no more than about 7.5 minutes, no more than about 5 minutes, no more than about 4 minutes, no more than about 3 minutes, no more than about 2 minutes, no more than about 1 minute, no more than 45 seconds, or no more than about 30 seconds.

If desired, the pharmaceutical composition formulation may further comprise a co-therapeutic agent. Co-administration of a co-therapeutic agent does not require that the co-therapeutic agent be included in the same pharmaceutical formulation as the TRP channel agonist. In some embodiments, the co-therapeutic agent is included in the pharmaceutical composition comprising the TRP channel agonist. In other embodiments, the co-therapeutic agent may be a separate pharmaceutical composition. Exemplary co-therapeutic agents may include, but are not limited to, mucoactive or mucolytic agents, surfactants, cough suppressants, expectorants, steroids, bronchodilators, antihistamines, anti-inflammatory agents, antibiotics, and antivirals. The co-therapeutic agents may be combined with other co-therapeutic agent(s) or with any of the TRP channel agonist(s) described herein.

Examples of suitable mucoactive or mucolytic agents include MUC5AC and MUC5B mucins, DNA-ase, N-acetylcysteine (NAC), cysteine, nacystelyn, dornase alfa, gelsolin, heparin, heparin sulfate, P2Y2 agonists (e.g. UTP, INS365), hypertonic saline, and mannitol.

Suitable surfactants include L-alpha-phosphatidylcholine dipalmitoyl (“DPPC”), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, phospholipids, and alkylated sugars.

Suitable cough suppressants include benzonatate, benproperine, clobutinal, diphenhydramine, dextromethorphan, dibunate, fedrilate, glaucine, oxalamine, piperidione, opiods such as codeine and the like.

Suitable expectorants include guaifenesin, guaiacolculfonate, ammonium chloride, potassium iodide, tyloxapol, antimony pentasulfide and the like.

Suitable steroids include corticosteroids, combinations of corticosteroids and long-acting beta₂ agonists (LABA), combinations of corticosteroids and long-acting muscarinic anagonists (LAMA), and the like. Suitable corticosteroids include budesonide, fluticasone, flunisolide, triamcinolone, beclomethasone, mometasone, ciclesonide, dexamethasone, and the like. Combinations of corticosteroids and LABAs include salmeterol with fluticasone, formoterol with budesonide, formoterol with fluticasone, formoterol with mometasone, indacaterol with mometasone, and the like.

Suitable bronchodilators include short-acting beta₂ agonists, long-acting beta₂ agonists (LABA), long-acting muscarinic anagonists (LAMA), combinations of LABAs and LAMAs, methylxanthines, and the like. Suitable short-active beta₂ agonists include albuterol, epinephrine, pirbuterol, levalbuterol, metaproteronol, maxair, and the like. Suitable LABAs include salmeterol, formoterol and isomers (e.g. arformoterol), clenbuterol, tulobuterol, vilanterol (Revolair™), indacaterol, and the like. Examples of LAMAs include tiotroprium, glycopyrrolate, aclidinium, ipratropium and the like. Examples of combinations of LABAs and LAMAs include indacaterol with glycopyrrolate, indacaterol with tiotropium, and the like. Examples of methylxanthine include theophylline, and the like.

Suitable antibiotics may include a macrolide (e.g., azithromycin, clarithromycin and erythromycin), a tetracycline (e.g., doxycycline, tigecycline), a fluoroquinolone (e.g., gemifloxacin, levofloxacin, ciprofloxacin and mocifloxacin), a cephalosporin (e.g., ceftriaxone, defotaxime, ceftazidime, cefepime), a penicillin (e.g., amoxicillin, amoxicillin with clavulanate, ampicillin, piperacillin, and ticarcillin) optionally with a β-lactamase inhibitor (e.g., sulbactam, tazobactam and clavulanic acid), such as ampicillin-sulbactam, piperacillin-tazobactam and ticarcillin with clavulanate, an aminoglycoside (e.g., amikacin, arbekacin, gentamicin, kanamycin, neomycin, netilmicin, paromomycin, rhodostreptomycin, streptomycin, tobramycin, and apramycin), a penem or carbapenem (e.g. doripenem, ertapenem, imipenem and meropenem), a monobactam (e.g., aztreonam), an oxazolidinone (e.g., linezolid), vancomycin, glycopeptide antibiotics (e.g. telavancin), tuberculosis-mycobacterium antibiotics and the like.

The antibiotic may be one for treating infections with mycobacteria, such as Mycobacterium tuberculosis. Suitable agents for treating infections with mycobacteria (e.g., M. tuberculosis) include an aminoglycoside (e.g. capreomycin, kanamycin, streptomycin), a fluoroquinolone (e.g. ciprofloxacin, levofloxacin, moxifloxacin), isozianid and isozianid analogs (e.g. ethionamide), aminosalicylate, cycloserine, diarylquinoline, ethambutol, pyrazinamide, protionamide, rifampin, and the like.

Suitable antiviral agents may include oseltamivir, zanamavir, amantidine, rimantadine, ribavirin, gancyclovir, valgancyclovir, foscavir, Cytogam® (Cytomegalovirus Immune Globulin), pleconaril, rupintrivir, palivizumab, motavizumab, cytarabine, docosanol, denotivir, cidofovir, and acyclovir. The salt formulation can contain a suitable anti-influenza agent, such as zanamivir, oseltamivir, amantadine, or rimantadine.

Suitable cough suppressants include benzonatate, benproperine, clobutinal, diphenhydramine, dextromethorphan, dibunate, fedrilate, glaucine, oxalamine, piperidione, opiods such as codeine and the like.

Suitable brochodilators include short-acting beta₂ agonists, long-acting beta₂ agonists (LABA), long-acting muscarinic anagonists (LAMA), combinations of LABAs and LAMAs, methylxanthines, short-acting anticholinergic agents (may also be referred to as short acting anti-muscarinic), long-acting bronchodilators and the like.

Suitable short-acting beta₂ agonists include albuterol, epinephrine, pirbuterol, levalbuterol, metaproteronol, maxair, and the like.

Examples of albuterol sulfate formulations (also called salbutamol) include Inspiryl (AstraZeneca Plc), Salbutamol SANDOZ (Sanofi-Aventis), Asmasal clickhaler (Vectura Group Plc.), Ventolin® (GlaxoSmithKline Plc), Salbutamol GLAND (GlaxoSmithKline Plc), Airomir® (Teva Pharmaceutical Industries Ltd.), ProAir HFA (Teva Pharmaceutical Industries Ltd.), Salamol (Teva Pharmaceutical Industries Ltd.), Ipramol (Teva Pharmaceutical Industries Ltd), Albuterol sulfate TEVA (Teva Pharmaceutical Industries Ltd), and the like. Examples of epinephrine include Epinephine Mist KING (King Pharmaceuticals, Inc.), and the like. Examples of pirbuterol as pirbuterol acetate include Maxair® (Teva Pharmaceutical Industries Ltd.), and the like. Examples of levalbuterol include Xopenex® (Sepracor), and the like. Examples of metaproteronol formulations as metaproteronol sulfate include Alupent® (Boehringer Ingelheim GmbH), and the like.

Suitable LABAs include salmeterol, formoterol and isomers (e.g. arformoterol), clenbuterol, tulobuterol, vilanterol (Revolair™), indacaterol, carmoterol, isoproterenol, procaterol, bambuterol, milveterol, olodaterol and the like.

Examples of salmeterol formulations include salmeterol xinafoate as Serevent® (GlaxoSmithKline Plc), salmeterol as Inaspir (Laboratorios Almirall, S.A.), Advair® HFA (GlaxoSmithKline PLC), Advair Diskus® (GlaxoSmithKline PLC, Theravance Inc), Plusvent (Laboratorios Almirall, S.A.), VR315 (Novartis, Vectura Group PLC) and the like. Examples of formoterol and isomers (e.g., arformoterol) include Foster (Chiesi Farmaceutici S.p.A), Atimos (Chiesi Farmaceutici S.p.A, Nycomed Internaional Management), Flutiform® (Abbott Laboratories, SkyePharma PLC), MFF258 (Novartis AG), Formoterol clickhaler (Vectura Group PLC), Formoterol HFA (SkyePharma PLC), Oxis® (Astrazeneca PLC), Oxis pMDI (Astrazeneca), Foradil® Aerolizer (Novartis, Schering-Plough Corp, Merck), Foradil® Certihaler (Novartis, SkyePharma PLC), Symbicort® (AstraZeneca), VR632 (Novartis AG, Sandoz International GmbH), MFF258 (Merck & Co Inc, Novartis AG), Alvesco® Combo (Nycomed International Management GmbH, Sanofi-Aventis, Sepracor Inc), Mometasone furoate (Schering-Plough Corp), and the like. Examples of clenbuterol include Ventipulmin® (Boehringer Ingelheim), and the like. Examples of tulobuterol include Hokunalin Tape (Abbott Japan Co., Ltd., Maruho Co., Ltd.), and the like. Examples of vilanterol include Revolair™ (GlaxoSmithKline PLC), GSK64244 (GlaxoSmithKline PLC), and the like. Examples of indacaterol include QAB149 (Novartis AG, SkyePharma PLC), QMF149 (Merck & Co Inc) and the like. Examples of carmoterol include CHF4226 (Chiese Farmaceutici S.p.A., Mitsubishi Tanabe Pharma Corporation), CHF5188 (Chiesi Farmaceutici S.p.A), and the like. Examples of isoproterenol sulfate include Aludrin (Boehringer Ingelheim GmbH) and the like. Examples of procaterol include Meptin clickhaler (Vectura Group PLC), and the like. Examples of bambuterol include Bambec (AstraZeneca PLC), and the like. Examples of milveterol include GSK159797C (GlaxoSmithKline PLC), TD3327 (Theravance Inc), and the like. Examples of olodaterol include BI1744CL (Boehringer Ingelheim GmbH) and the like.

Examples of LAMAs include tiotroprium, trospium chloride, glycopyrrolate, aclidinium, ipratropium and the like.

Examples of tiotroprium include Spiriva (Boehringer-Ingleheim, Pfizer), and the like. Examples of glycopyrrolate include Robinul® (Wyeth-Ayerst), Robinul® Forte (Wyeth-Ayerst), NVA237 (Novartis), and the like. Examples of aclidinium include Eklira® (Forest Labaoratories, Almirall), and the like.

Examples of combinations of LABAs and LAMAs include indacaterol with glycopyrrolate, formoterol with glycopyrrolate, indacaterol with tiotropium, olodaterol and tiotropium, vilanterol with a LAMA, and the like.

Examples of combinations of indacaterol with glycopyrrolate include QVA149A (Novartis), and the like. Examples of combinations of formoterol with glycopyrrolate include PT003 (Pearl Therapeutics) and the like. Examples of combinations of olodaterol with tiotropium include BI1744 with Spirva (Boehringer Ingelheim) and the like. Examples of combinations of vilanterol with a LAMA include GSK573719 with GSK642444 (GlaxoSmithKline PLC), and the like.

Examples of methylxanthine include aminophylline, ephedrine, theophylline, oxtriphylline, and the like.

Examples of aminophylline include Aminophylline BOEHRINGER (Boehringer Ingelheim GmbH) and the like. Examples of ephedrine include Bronkaid® (Bayer AG), Broncholate (Sanofi-Aventis), Primatene® (Wyeth), Tedral SA®, Marax (Pfizer Inc) and the like. Examples of theophylline include Euphyllin (Nycomed International Management GmbH), Theo-dur (Pfizer Inc, Teva Pharmacetuical Industries Ltd) and the like. Examples of oxtriphylline include Choledyl SA (Pfizer Inc) and the like.

Examples of short-acting anticholinergic agents include ipratropium bromide, oxitropium bromide, and tiotropium (Spiriva).

Examples of ipratropium bromide include Atrovent/Apovent/Inpratropio (Boehringer Ingelheim GmbH), Ipramol (Teva Pharmaceutical Industries Ltd) and the like. Examples of oxitropium bromide include Oxivent (Boehringer Ingelheim GmbH), and the like.

Suitable anti-inflammatory agents include leukotriene inhibitors, phosphodiesterase 4 (PDE4) inhibitors, other anti-inflammatory agents, and the like.

Suitable leukotriene inhibitors include montelukast (cystinyl leukotriene inhibitors), masilukast, zafirleukast (leukotriene D4 and E4 receptor inhibitors), pranlukast, zileuton (5-lipoxygenase inhibitors), and the like.

Examples of montelukast (cystinyl leukotriene inhibitor) include Singulair (Merck & Co Inc), Loratadine, montelukast sodium SCHERING (Schering-Plough Corp), MK0476C (Merck & Co Inc), and the like. Examples of masilukast include MCC847 (AstraZeneca PLC), and the like. Examples of zafirlukast (leukotriene D4 and E4 receptor inhibitor) include Accolate® (AstraZeneca PLC), and the like. Examples of pranlukast include Azlaire (Schering-Plough Corp). Examples of zileuton (5-LO) include Zyflo® (Abbott Laboratories), Zyflo CR® (Abbott Laboratories, SkyePharma PLC), Zileuton ABBOTT LABS (Abbott Laboratories), and the like. Suitable PDE4 inhibitors include cilomilast, roflumilast, oglemilast, tofimilast, and the like.

Examples of cilomilast include Ariflo (GlaxoSmithKline PLC), and the like. Examples of roflumilast include Daxas® (Nycomed International Management GmbH, Pfizer Inc), APTA2217 (Mitsubishi Tanabe Pharma Corporation), and the like. Examples of oglemilast include GRC3886 (Forest Laboratories Inc), and the like. Examples of tofimilast include Tofimilast PFIZER INC (Pfizer Inc), and the like.

Other anti-inflammatory agents include omalizumab (anti-IgE immunoglobulin Daiichi Sankyo Company, Limited), Zolair (anti-IgE immunoglobulin, Genentech Inc, Novartis AG, Roche Holding Ltd), Solfa (LTD4 antagonist and phosphodiesterase inhibitor, Takeda Pharmaceutical Company Limited), IL-13 and IL-13 receptor inhibitors (such as AMG-317, MILR1444A, CAT-354, QAX576, IMA-638, Anrukinzumab, IMA-026, MK-6105, DOM-0910, and the like), IL-4 and IL-4 receptor inhibitors (such as Pitrakinra, AER-003, AIR-645, APG-201, DOM-0919, and the like), IL-1 inhibitors such as canakinumab, CRTh2 receptor antagonists such as AZD1981 (CRTh2 receptor antagonist, AstraZeneca), neutrophil elastase inhibitor such as AZD9668 (neutrophil elastase inhibitor, from AstraZeneca), GW856553X Losmapimod (P38 kinase inhibitor, GlaxoSmithKline PLC), Arofylline LAB ALMIRALL (PDE-4 inhibitor, Laboratorios Almirall, S.A.), ABT761 (5-LO inhibitor, Abbott Laboratories), Zyflo® (5-LO inhibitor, Abbott Laboratories), BT061 (anti-CD4 mAb, Boehringer Ingelheim GmbH), Corns (inhaled lidocaine to decrease eosinophils, Gilead Sciences Inc), Prograf (IL-2-mediated T-cell activation inhibitor, Astellas Pharma), Bimosiamose PFIZER INC (selectin inhibitor, Pfizer Inc), R411 (α4β1/α4β7 integrin antagonist, Roche Holdings Ltd), Tilade® (inflammatory mediator inhibitor, Sanofi-Aventis), Orenica® (T-cell co-stimulation inhibitor, Bristol-Myers Squibb Company), Soliris® (anti-05, Alexion Pharmaceuticals Inc), Entorken® (Farmacija d.o.o.), Excellair® (Syk kinase siRNA, ZaBeCor Pharmaceuticals, Baxter International Inc), KB003 (anti-GMCSF mAb, KaloBios Pharmaceuticals), Cromolyn sodiums (inhibit release of mast cell mediators): Cromolyn sodium BOEHRINGER (Boehringer Ingelheim GmbH), Cromolyn sodium TEVA (Teva Pharmaceutical Industries Ltd), Intal (Sanofi-Aventis), BI1744CL (oldaterol (β2-adrenoceptor antagonist) and tiotropium, Boehringer Ingelheim GmbH), NFκ-B inhibitors, CXR2 antagaonists, HLE inhibitors, HMG-CoA reductase inhibitors and the like.

Anti-inflammatory agents also include compounds that inhibit/decrease cell signaling by inflammatory molecules like cytokines (e.g., IL-1, IL-4, IL-5, IL-6, IL-9, IL-13, IL-18 IL-25, IFN-α, IFN-β, and others), CC chemokines CCL-1-CCL28 (some of which are also known as, for example, MCP-1, CCL2, RANTES), CXC chemokines CXCL1-CXCL17 (some of which are also know as, for example, IL-8, MIP-2), growth factors (e.g., GM-CSF, NGF, SCF, TGF-β, EGF, VEGF and others) and/or their respective receptors.

Some examples of the aforementioned anti-inflammatory antagonists/inhibitors include ABN912 (MCP-1/CCL2, Novartis AG), AMG761 (CCR4, Amgen Inc), Enbrel® (TNF, Amgen Inc, Wyeth), huMAb OX40L GENENTECH (TNF superfamily, Genentech Inc, AstraZeneca PLC), R4930 (TNF superfamily, Roche Holding Ltd), SB683699/Firategrast (VLA4, GlaxoSmithKline PLC), CNT0148 (TNFα, Centocor, Inc, Johnson & Johnson, Schering-Plough Corp); Canakinumab (IL-1β, Novartis); Israpafant MITSUBISHI (PAF/IL-5, Mitsubishi Tanabe Pharma Corporation); IL-4 and IL-4 receptor antagonists/inhibitors: AMG317 (Amgen Inc), BAY169996 (Bayer AG), AER-003 (Aerovance), APG-201 (Apogenix); IL-5 and IL-5 receptor antagonists/inhibitors: MEDI563 (AstraZeneca PLC, Medlmmune, Inc), Bosatria® (GlaxoSmithKline PLC), Cinquil® (Ception Therapeutic), TMC120B (Mitsubishi Tanabe Pharma Corporation), Bosatria (GlaxoSmithKline PLC), Reslizumab SCHERING (Schering-Plough Corp); MEDI528 (IL-9, AstraZeneca, MedImmune, Inc); IL-13 and IL-13 receptor antagonists/inhibitors: TNX650 GENETECH (Genetech), CAT-354 (AstraZeneca PLC, MedImmune), AMG-317 (Takeda Pharmaceutical Company Limited), MK6105 (Merck & Co Inc), IMA-026 (Wyeth), IMA-638 Anrukinzumab (Wyeth), MILR1444A/Lebrikizumab (Genentech), QAX576 (Novartis), CNTO-607 (Centocor), MK-6105 (Merck, CSL); Dual IL-4 and IL-13 inhibitors: AIR645/ISIS369645 (ISIS Altair), DOM-0910 (GlaxoSmithKline, Domantis), Pitrakinra/AER001/Aerovant™ (Aerovance Inc), AMG-317 (Amgen), and the like.

Suitable steroids include corticosteroids, combinations of corticosteroids and LABAs, combinations of corticosteroids and LAMAs, combinations of corticosteroids, LABAs and LAMAs, and the like.

Suitable corticosteroids include budesonide, fluticasone, flunisolide, triamcinolone, beclomethasone, mometasone, ciclesonide, dexamethasone, and the like.

Examples of budesonide include Captisol-Enabled Budesonide Solution for Nebulization (AstraZeneca PLC), Pulmicort® (AstraZeneca PLC), Pulmicort® Flexhaler (AstraZeneca Plc), Pulmicort® HFA-MDI (AstraZeneca PLC), Pulmicort Respules® (AstraZeneca PLC), Inflammide (Boehringer Ingelheim GmbH), Pulmicort® HFA-MDI (SkyePharma PLC), Unit Dose Budesonide ASTRAZENECA (AstraZeneca PLC), Budesonide Modulite (Chiesi Farmaceutici S.p.A), CHF5188 (Chiesi Farmaceutici S.p.A), Budesonide ABBOTT LABS (Abbott Laboratories), Budesonide clickhaler (Vestura Group PLC), Miflonide (Novartis AG), Xavin (Teva Pharmaceutical Industries Ltd.), Budesonide TEVA (Teva Pharmaceutical Industries Ltd.), Symbicort® (AstraZeneca K.K., AstraZeneca PLC), VR632 (Novartis AG, Sandoz International GmbH), and the like.

Examples of fluticasone propionate formulations include Flixotide Evohaler (GlaxoSmithKline PLC), Flixotide Nebules (GlaxoSmithKline Plc), Flovent® (GlaxoSmithKline Plc), Flovent® Diskus (GlaxoSmithKline PLC), Flovent® HFA (GlaxoSmithKline PLC), Flovent® Rotadisk (GlaxoSmithKline PLC), Advair® HFA (GlaxoSmithKline PLC, Theravance Inc), Advair Diskus® (GlaxoSmithKline PLC, Theravance Inc.), VR315 (Novartis AG, Vectura Group PLC, Sandoz International GmbH), and the like. Other formulations of fluticasone include fluticasone as Flusonal (Laboratorios Almirall, S.A.), fluticasone furoate as GW685698 (GlaxoSmithKline PLC, Thervance Inc.), Plusvent (Laboratorios Almirall, S.A.), Flutiform® (Abbott Laboratories, SkyePharma PLC), and the like.

Examples of flunisolide include Aerobid® (Forest Laboratories Inc), Aerospan® (Forest Laboratories Inc), and the like. Examples of triamcinolone include Triamcinolone ABBOTT LABS (Abbott Laboratories), Azmacort® (Abbott Laboratories, Sanofi-Aventis), and the like. Examples of beclomethasone dipropionate include Beclovent (GlaxoSmithKline PLC), QVAR® (Johnson & Johnson, Schering-Plough Corp, Teva Pharmacetucial Industries Ltd), Asmabec clickhaler (Vectura Group PLC), Beclomethasone TEVA (Teva Pharmaceutical Industries Ltd), Vanceril (Schering-Plough Corp), BDP Modulite (Chiesi Farmaceutici S.p.A.), Clenil (Chiesi Farmaceutici S.p.A), Beclomethasone dipropionate TEVA (Teva Pharmaceutical Industries Ltd), and the like. Examples of mometasone include QAB149 Mometasone furoate (Schering-Plough Corp), QMF149 (Novartis AG), Fomoterol fumarate, mometoasone furoate (Schering-Plough Corp), MFF258 (Novartis AG, Merck & Co Inc), Asmanex® Twisthaler (Schering-Plough Corp), and the like. Examples of cirlesonide include Alvesco® (Nycomed International Management GmbH, Sepracor, Sanofi-Aventis, Tejin Pharma Limited), Alvesco® Combo (Nycomed International Management GmbH, Sanofi-Aventis), Alvesco® HFA (Nycomed Intenational Management GmbH, Sepracor Inc), and the like. Examples of dexamethasone include DexPak® (Merck), Decadron® (Merck), Adrenocot, CPC-Cort-D, Decaject-10, Solurex and the like. Other corticosteroids include Etiprednol dicloacetate TEVA (Teva Pharmaceutical Industries Ltd), and the like.

Combinations of corticosteroids and LABAs include salmeterol with fluticasone, formoterol with budesonide, formoterol with fluticasone, formoterol with mometasone, indacaterol with mometasone, and the like.

Examples of salmeterol with fluticasone include Plusvent (Laboratorios Almirall, S.A.), Advair® HFA (GlaxoSmithKline PLC), Advair® Diskus (GlaxoSmithKline PLV, Theravance Inc), VR315 (Novartis AG, Vectura Group PLC, Sandoz International GmbH) and the like. Examples of vilanterol with fluticasone include GSK642444 with fluticasone and the like. Examples of formoterol with budesonide include Symbicort® (AstraZeneca PLC), VR632 (Novartis AG, Vectura Group PLC), and the like. Examples of formoterol with fluticasone include Flutiform® (Abbott Laboratories, SkyePharma PLC), and the like. Examples of formoterol with mometasone include Dulera®/MFF258 (Novartis AG, Merck & Co Inc), and the like. Examples of indacaterol with mometasone include QAB149 Mometasone furoate (Schering-Plough Corp), QMF149 (Novartis AG), and the like. Combinations of corticosteroids with LAMAs include fluticasone with tiotropium, budesonide with tiotropium, mometasone with tiotropium, salmeterol with tiotropium, formoterol with tiotropium, indacaterol with tiotropium, vilanterol with tiotropium, and the like. Combinations of corticosteroids with LAMAs and LABAs include fluticasone with salmeterol and tiotropium.

Other anti-asthma molecules include: ARD111421 (VIP agonist, AstraZeneca PLC), AVE0547 (anti-inflammatory, Sanofi-Aventis), AVE0675 (TLR agonist, Pfizer, Sanofi-Aventis), AVE0950 (Syk inhibitor, Sanofi-Aventis), AVE5883 (NK1/NK2 antagonist, Sanofi-Aventis), AVE8923 (tryptase beta inhibitor, Sanofi-Aventis), CGS21680 (adenosine A2A receptor agonist, Novartis AG), ATL844 (A2B receptor antagonist, Novartis AG), BAY443428 (tryptase inhibitor, Bayer AG), CHF5407 (M3 receptor inhibitor, Chiesi Farmaceutici S.p.A.), CPLA2 Inhibitor WYETH (CPLA2 inhibitor, Wyeth), IMA-638 (IL-13 antagonist, Wyeth), LAS100977 (LABA, Laboratorios Almirall, S.A.), MABA (M3 and β2 receptor antagonist, Chiesi Farmaceutici S.p.A), R1671 (mAb, Roche Holding Ltd), CS003 (Neurokinin receptor antagonist, Daiichi Sankyo Company, Limited), DPC168 (CCR antagonist, Bristol-Myers Squibb), E26 (anti-IgE, Genentech Inc), HAE1 (Genentech), IgE inhibitor AMGEN (Amgen Inc), AMG853 (CRTH2 and D2 receptor antagonist, Amgen), IPL576092 (LSAID, Sanofi-Aventis), EPI2010 (antisense adenosine 1, Chiesi Farmaceutici S.p.A.), CHF5480 (PDE-4 inhibitor, Chiesi Farmaceutici S.p.A.), KI04204 (corticosteroid, Abbott Laboratories), SVT47060 (Laboratorios Salvat, S.A.), VML530 (leukotriene synthesis inhibitor, Abbott Laboratories), LAS35201 (M3 receptor antagonist, Laboratorios Almirall, S.A.), MCC847 (D4 receptor antagonist, Mitsubishi Tanabe Pharma Corporation), MEM1414 (PDE-4 inhibitor, Roche), TA270 (5-LO inhibitor, Chugai Pharmaceutical Co Ltd), TAK661 (eosinophil chemotaxis inhibitor, Takeda Pharmaceutical Company Limited), TBC4746 (VLA-4 antagonist, Schering-Plough Corp), VR694 (Vectura Group PLC), PLD177 (steroid, Vectura Group PLC), KI03219 (corticosteroid+LABA, Abbott Laboratories), AMG009 (Amgen Inc), AMG853 (D2 receptor antagonist, Amgen Inc);

AstraZeneca PLC: AZD1744 (CCR3/histamine-1 receptor antagonist, AZD1419 (TLR9 agonist), Mast Cell inhibitor ASTRAZENECA, AZD3778 (CCR antagonist), DSP3025 (TLR7 agonist), AZD1981 (CRTh2 receptor antagonist), AZD5985 (CRTh2 antagonist), AZD8075 (CRTh2 antagonist), AZD1678, AZD2098, AZD2392, AZD3825 AZD8848, AZD9215, ZD2138 (5-LO inhibitor), AZD3199 (LABA);

GlaxoSmithKline PLC: GW328267 (adenosine A2 receptor agonist), GW559090 (α4 integrin antagonist), GSK679586 (mAb), GSK597901 (adrenergic β2 agonist), AM103 (5-LO inhibitor), GSK256006 (PDE4 inhibitor), GW842470 (PDE-4 inhibitor), GSK870086 (glucocorticoid agonist), GSK159802 (LABA), GSK256066 (PDE-4 inhibitor), GSK642444 (LABA, adrenergic β2 agonist), GSK64244 and Revolair (fluticasone/vilanterol), GSK799943 (corticosteroid), GSK573719 (mAchR antagonist), and GSK573719.

Pfizer Inc: PF3526299, PF3893787, PF4191834 (FLAP antagonist), PF610355 (adrenergic β2 agonist), CP664511 (α4β1/VCAM-1 interaction inhibitor), CP609643 (inhibitor of α4β1/VCAM-1 interactions), CP690550 (JAK3 inhibitor), SAR21609 (TLR9 agonist), AVE7279 (Th1 switching), TBC4746 (VLA-4 antagonist); R343 (IgE receptor signaling inhibitor), SEP42960 (adenosine A3 antagonist);

Sanofi-Aventis: MLN6095 (CrTH2 inhibitor), SAR137272 (A3 antagonist), SAR21609 (TLR9 agonist), SAR389644 (DP1 receptor antagonist), SAR398171 (CRTH2 antagonist), SSR161421 (adenosine A3 receptor antagonist);

Merck & Co Inc: MK0633, MK0633, MK0591 (5-LO inhibitor), MK886 (leukotriene inhibitor), BIO1211 (VLA-4 antagonist); Novartis AG: QAE397 (long-acting corticosteroid), QAK423, QAN747, QAP642 (CCR3 antagonist), QAX935 (TLR9 agonist), NVA237 (LAMA).

Suitable expectorants include guaifenesin, guaiacolculfonate, ammonium chloride, potassium iodide, tyloxapol, antimony pentasulfide and the like.

Suitable vaccines include nasally inhaled influenza vaccines and the like.

Suitable macromolecules include proteins and large peptides, polysaccharides and oligosaccharides, and DNA and RNA nucleic acid molecules and their analogs having therapeutic, prophylactic or diagnostic activities. Proteins can include antibodies such as monoclonal antibodies. Nucleic acid molecules include genes, antisense molecules such as siRNAs that bind to complementary DNA, RNAi, shRNA, microRNA, RNA, or ribosomes to inhibit transcription or translation. Preferred macromolecules have a molecular weight of at least 800 Da, at least 3000 Da or at least 5000 Da.

Selected macromolecule drugs for include Ventavis® (Iloprost), Calcitonin, Erythropoietin (EPO), Factor IX, Granulocyte Colony Stimulating Factor (G-CSF), Granulocyte Macrophage Colony, Stimulating Factor (GM-CSF), Growth Hormone, Insulin, Interferon Alpha, Interferon Beta, Interferon Gamma, Luteinizing Hormone Releasing Hormone (LHRH), follicle stimulating hormone (FSH), Ciliary Neurotrophic Factor, Growth Hormone Releasing Factor (GRF), Insulin-Like Growth Factor, Insulinotropin, Interleukin-1 Receptor Antagonist, Interleukin-3, Interleukin-4, Interleukin-6, Macrophage Colony Stimulating Factor (M-CSF), Thymosin Alpha 1, IIb/IIIa Inhibitor, Alpha-1 Antitrypsin, Anti-RSV Antibody, palivizumab, motavizumab, and ALN-RSV, Cystic Fibrosis Transmembrane Regulator (CFTR) Gene, Deoxyribonuclase (DNase), Heparin, Bactericidal/Permeability Increasing Protein (BPI), Anti-Cytomegalovirus (CMV) Antibody, Interleukin-1 Receptor Antagonist, and the like. GLP-1 analogs (liraglutide, exenatide, etc.), Domain antibodies (dAbs), Pramlintide acetate (Symlin), Leptin analogs, Synagis (palivizumab, MedImmune) and cisplatin.

Selected therapeutics helpful for chronic maintenance of CF include antibiotics/macrolide antibiotics, bronchodilators, inhaled LABAs, and agents to promote airway secretion clearance. Suitable examples of antibiotics/macrolide antibiotics include tobramycin, azithromycin, ciprofloxacin, colistin, aztreonam and the like. Suitable examples of bronchodilators include inhaled short-acting beta₂ agonists such as albuterol, and the like. Suitable examples of inhaled LABAs include salmeterol, formoterol, and the like. Suitable examples of agents to promote airway secretion clearance include Pulmozyme (dornase alfa, Genetech), hypertonic saline, DNase, heparin and the like. Selected therapeutics helpful for the prevention and/or treatment of CF include VX-770 (Vertex Pharmaceuticals) and amiloride.

TABLE 1
Agonists of TRP Channels
TRPA1 Agonists

| | |
|---|---|
| 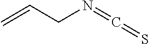 | Allyl isothiocyanate (AITC) (Mustard oil) |
| 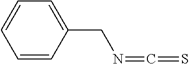 | Benyzl isothiocyanate (BITC) |
| 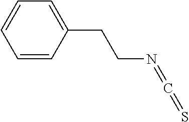 [SMILES: S=C=NCCc1ccccc1] | Phenyl isothiocyanate |
| 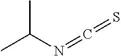 | Isopropyl isothiocyanate |
| 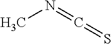 | Methyl isothiocyanate |
| 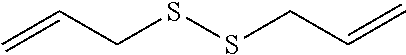 | Diallyl disulfide |
| 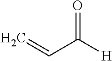 | Acrolein (2-propenal) |
| 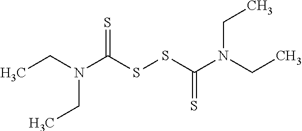 [SMILES: CCN(CC)C(=S)SSC(=S)N(CC)CC] | Disulfiram (Antabuse ®, Odyssey Pharmaceuticals) |
| 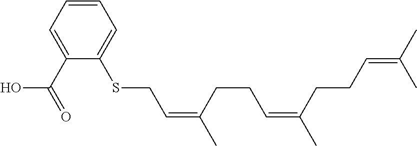 [SMILES: CC(C)=CCC/C(C)=C\CC/C(C)=C\CSc1ccccc1C(=O)O] | Farnesyl thiosalicylic acid (FTS) |
| 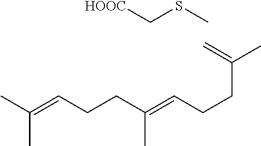 | Farnesyl thioacetic acid (FTA) |
| 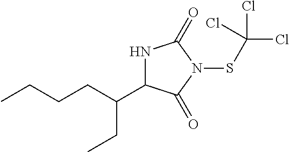 [SMILES: CCCCC(CC)C1NC(=O)N(SC(Cl)(Cl)Cl)C1=O] | Chlodantoin (Sporostacin ®) |
| 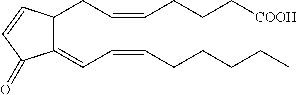 [SMILES: CCCCC/C=C\C=C1\C(=O)C=CC1C/C=C\CCCC(=O)O] | 15-deoxy-Δ-12,14-PGJ₂ (15d-PGJ2) |
| 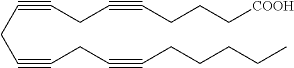 [SMILES: CCCCCC#CCC#CCC#CCC#CCCCC(=O)O] | 5, 8, 11, 14 Eicosatetraynoic acid (ETYA) |
| 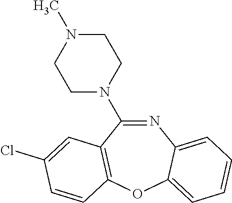 [SMILES: CN1CCN(C2=Nc3ccccc3Oc3ccc(Cl)cc32)CC1] | Dibenzoazepine (WO9747611) |
| 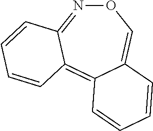 [SMILES: C1=c2ccccc2=c2ccccc2=NO1] | Dibenzoxazepine (WO9747611) |
| 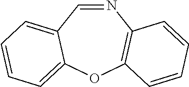 [SMILES: C1=Nc2ccccc2Oc2ccccc21] | Dibenz[b,f]-[1,4]oxazepine (CR) |
| 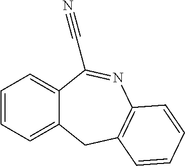 [SMILES: N#CC1=Nc2ccccc2Cc2ccccc21] | 11 H-dibenz[b,e]azepine |
| 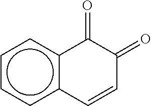 | 1,2 naphthoquione |
| 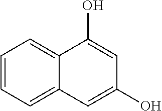 [SMILES: Oc1cc(O)c2ccccc2c1] | 1,3-dihydroxynaphthalene |
| 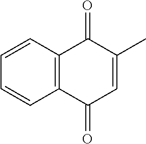 | 2 methyl-1,4-naphthoquinone |
| 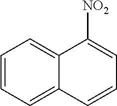 [SMILES: O=[N+]([O-])c1cccc2ccccc12] | 1-nitronaphthalene |
| 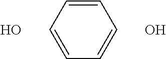 | hydroquinone |
| 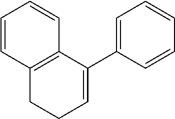 [SMILES: C1=C(c2ccccc2)c2ccccc2CC1] | 4-phenyl-1,2-dihydronaphthalene |
| 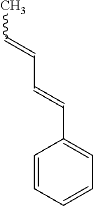 | 4-phenyl-1,2-dihydronaphthalene |
| 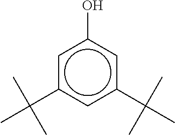 | 3,5-ditert-butylphenol |
| 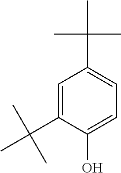 | 2,4-ditert-butylphenol |
| 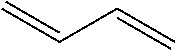 | 1,3 butadiene |
| [(3E)-1-phenyl-1,3-pentadienyl]benzene | |
| [(2Z)-3-phenyl-2-butenyl]benzene | |
| 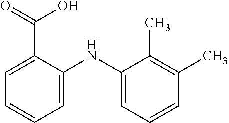 [SMILES: Cc1cccc(Nc2ccccc2C(=O)O)c1C] | Mefenamic acid |
| 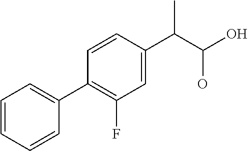 [SMILES: CC(c1ccc(-c2ccccc2)c(F)c1)C(O)O] | Fluribiprofen |
| 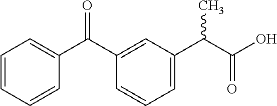 [SMILES: CC(C(=O)O)c1cccc(C(=O)c2ccccc2)c1] | Ketoprofen |
| 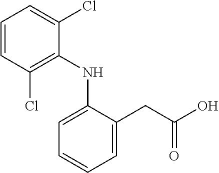 [SMILES: O=C(O)Cc1ccccc1Nc1c(Cl)cccc1Cl] | Diclofenac |
| 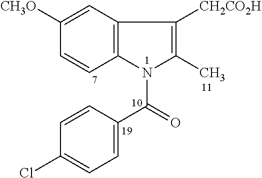 | Indomethacin |
| 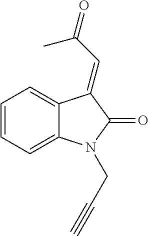 [SMILES: C#CCN1C(=O)/C(=C/C(C)=O)c2ccccc21] | SC alkyne (SCA) |
| 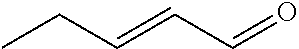 | Pentenal |
| 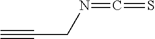 | Mustard oil alkyne (MOA) |
| 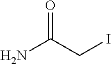 | Iodoacetamide |
| 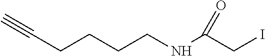 | Iodoacetamide alkyne |
| 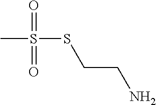 | (2-aminoethyl) methanethiosulphonate (MTSEA) |
| 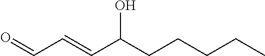 | 4-hydroxy-2-noneal (HNE) |
| 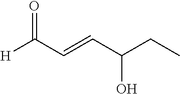 | 4-hydroxy xexenal (HHE) |
| 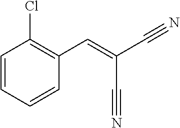 [SMILES: N#CC(C#N)=Cc1ccccc1Cl] | 2-chlorobenzalmalononitrile |
| 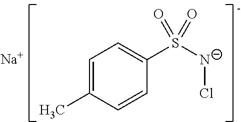 | N-chloro tosylamide (chloramine-T) |
| 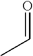 | Aldehyde |
| 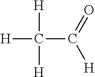 | Acetaldehyde (US2009269280) |
| 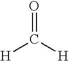 | Formaldehyde |
| 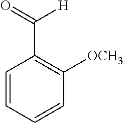 | O-anisaldehyde |
| 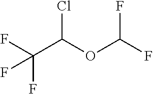 | Isoflurane |
| 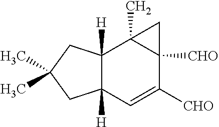 | Isovelleral |
| 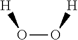 | Hydrogen peroxide |
| 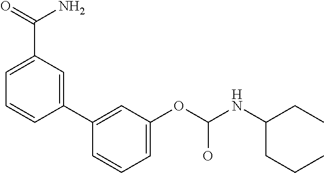 [SMILES: CCCC(CC)NC(O)Oc1cccc(-c2cccc(C(N)=O)c2)c1] | URB597 |
| 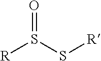 | Thiosulfinate |
| 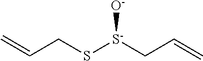 | Allicin |
| 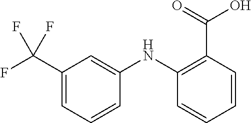 [SMILES: O=C(O)c1ccccc1Nc1cccc(C(F)(F)F)c1] | Flufenamic acid |
| 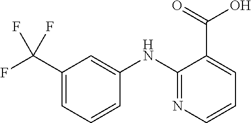 [SMILES: O=C(O)c1cccnc1Nc1cccc(C(F)(F)F)c1] | Niflumic acid |
| 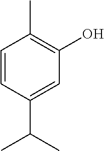 | Carvacrol |
| 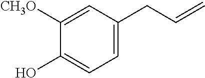 | Eugenol |
| 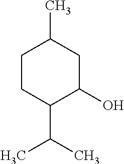 | Menthol |
| 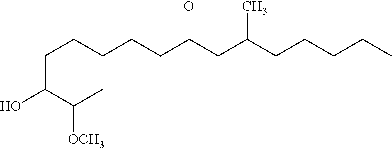 | Gingerol |
| 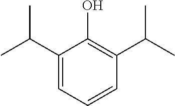 | Propofol |
| 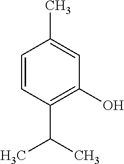 | Thymol |
| 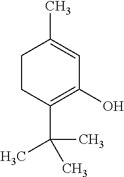 | 2-tert-butyl-5-methylphenol |
| 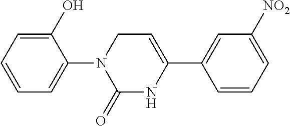 [SMILES: O=C1NC(c2cccc([N+](=O)[O-])c2)=CCN1c1ccccc1O] | Icilin |
| 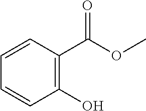 | Methyl salicylate |
| 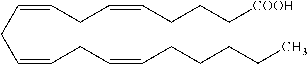 [SMILES: CCCCC/C=C\C/C=C\C/C=C\C/C=C\CCCC(=O)O] | Arachidonic acid |
| 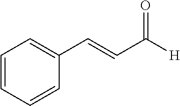 | Cinnemaldehyde |
| 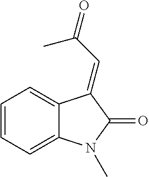 | Super Cinnemaldehyde |
| 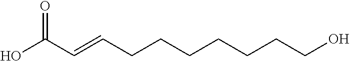 [SMILES: O=C(O)/C=C/CCCCCCCO] | 10-hydroxy-2-decenoic acid |
| 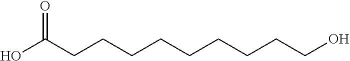 [SMILES: O=C(O)CCCCCCCCCO] | 10-hydroxydecanoic acid |
| 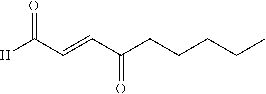 | 4-oxo-2-nonenal (4-ONE) |
| 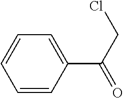 | 1-chloroacetophenone (CN) |
| 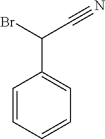 | Bromobenzyl cyanide |

Compounds CA3, 13-19 and 21-27 from Defalco J et al., 2010, Bioorg Med Chem Lett. 20(1): 276-279.

| | |
|---|---|
| 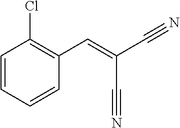 [SMILES: N#CC(C#N)=Cc1ccccc1Cl] | 2-chlorobenzylidene malononitrile (CS) |
| 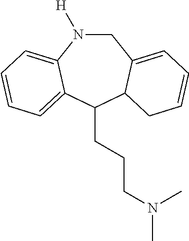 [SMILES: CN(C)CCCC1c2ccccc2NCC2=CC=CCC21] | morphanthridine |

Compounds 6 and 32 from Gijsen HJ et al., 2010, J Med Chem 53(19):7011-702.

| | |
|---|---|
| 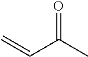 | methyl vinyl ketone |
| 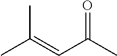 | mesityl oxide |
| 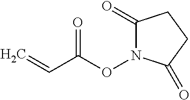 | Acrylic acid N-hydroxysuccinimide ester |
| 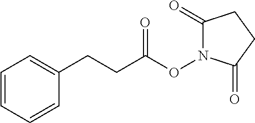 [SMILES: O=C(CCc1ccccc1)ON1C(=O)CCC1=O] | Hydrocinnamic acid N-hydroxysuccinimide ester |
| 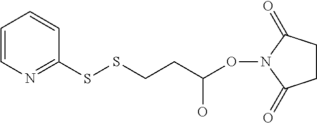 [SMILES: O=C1CCC(=O)N1OC(O)CCSSc1ccccn1] | 3-(2-Pyridyldithio)propionic acid N- hydroxysuccinimide ester |
| 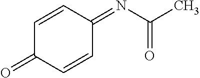 | N-acetyl-p-benzo-quinoneimine |
| 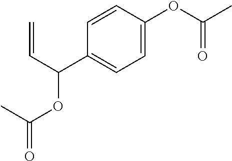 [SMILES: C=CC(OC(C)=O)c1ccc(OC(C)=O)cc1] | 1′-acetoxychavicol acetate |
| 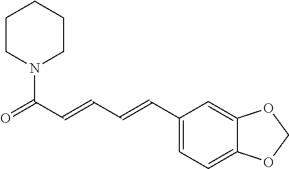 [SMILES: O=C(/C=C/C=C/c1ccc2c(c1)OCO2)N1CCCCC1] | Piperine |
| 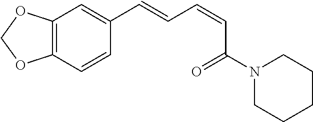 [SMILES: O=C(/C=C\C=C\c1ccc2c(c1)OCO2)N1CCCCC1] | Isopiperine |
| 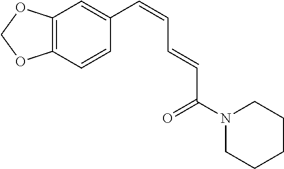 [SMILES: O=C(/C=C/C=C\c1ccc2c(c1)OCO2)N1CCCCC1] | Isochavicine |
| 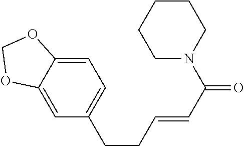 [SMILES: O=C(/C=C/CCc1ccc2c(c1)OCO2)N1CCCCC1] | Piperanine |
| 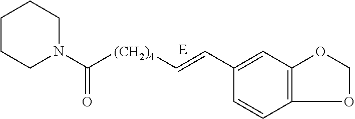 | Piperolein A |
| 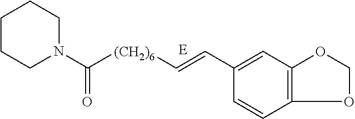 | Piperolein B |
| 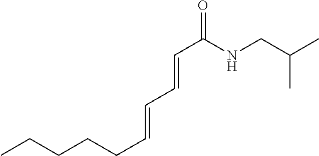 [SMILES: CCCCC/C=C/C=C/C(=O)NCC(C)C] | (2E,4E)-N-Isobuty1-2,4-decadienamide |
| 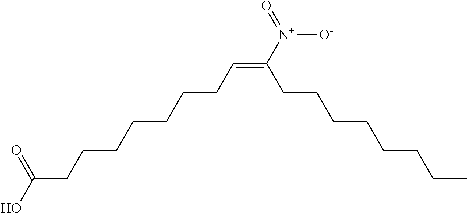 [SMILES: CCCCCCCC/C(=C\CCCCCCCC(=O)O)[N+](=O)[O-]] | Nitro-oleic acid (OA—NO₂) |
| 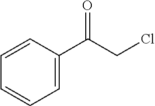 | 2-chloroacetophenone |
| 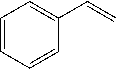 | Styrene |
| 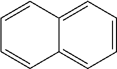 | Naphthalene |
| 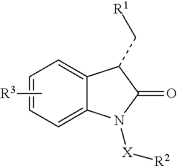 | Indolinone compounds (US2011009379) |
| 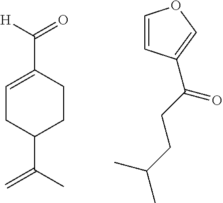 | Compounds from Perilla frutescens. Bassoli et al., 2009, Biorg Med Chem 17(4): 1636-1639. |

Cannabinoids

| | |
|---|---|
| 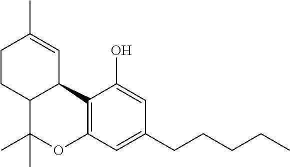 | Tetrahydrocannabinol (THC or Δ⁹-THC) |
| 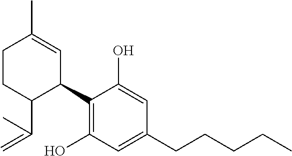 [SMILES: C=C(C)C1CCC(C)=C[C@H]1c1c(O)cc(CCCCC)cc1O] | Cannabidiol (CBD) |
| 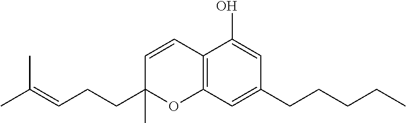 [SMILES: CCCCCc1cc(O)c2c(c1)OC(C)(CCC=C(C)C)C=C2] | Cannabichromene (CBC) |
| 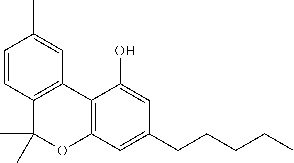 [SMILES: CCCCCc1cc(O)c2c(c1)OC(C)(C)c1ccc(C)cc1-2] | Cannabinol (CBN) |
| 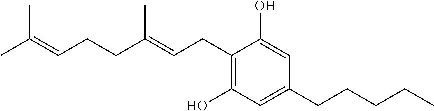 [SMILES: CCCCCc1cc(O)c(C/C=C(\C)CCC=C(C)C)c(O)c1] | Cannabigerol (CBG) |
| 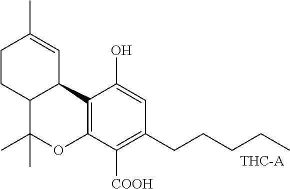 | THC acid (THC-A) |
| 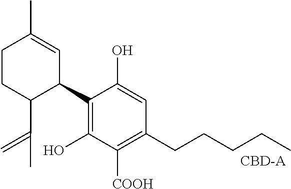 [SMILES: C=C(C)C1CCC(C)=C[C@H]1c1c(O)cc(CCCCC)c(C(=O)O)c1O] | CBD acid (CBD-A) |
| 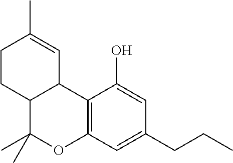 [SMILES: CCCc1cc(O)c2c(c1)OC(C)(C)C1CCC(C)=CC21] | Tetrahydrocannabivarin (THCVA) |

Activates rat TRPA1, antagonizes human TRPA1

| | |
|---|---|
| 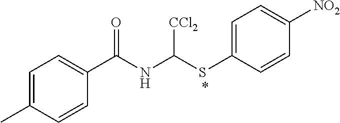 | Compound 1 from Chen et al., 2008, J Neuroscience 28(19): 5063-5071. (AMG5445) |
| 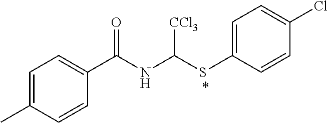 | 4-methyl-N-[2,2,2-trichloro-1-(4- chlorophenylsulfanyl)ethyl]benzamide (Abbott). Compound 2 from Chen et al., 2008, J Neuroscience 28(19): 5063-5071. |
| 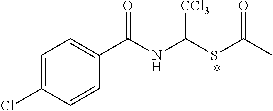 | N-[2,2,2-trichloro-1-(4-chlorophenylsulfanyl) ethyl]acetamide (Abbott). Compound 3 from Chen et al., 2008, J Neuroscience 28(19): 5063- 5071. |
| 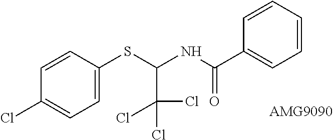 [SMILES: O=C(NC(Sc1ccc(Cl)cc1)C(Cl)(Cl)Cl)c1ccccc1] | AMG9090 (Amgen) |
| 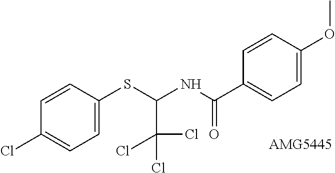 [SMILES: COc1ccc(C(=O)NC(Sc2ccc(Cl)cc2)C(Cl)(Cl)Cl)cc1] | AMG5445 (Amgen) |

### EXEMPLIFICATION

### Methods

Cell culture models of influenza, rhinovirus or parainfluenza infection were used to study the role of TRP channels in calcium-sodium formulation inhibition of viral infection. Calu-3 cells (American Type Culture Collection, Manassas, Va.) were cultured on permeable membranes (12 mm Transwells, 0.4 μm pore size; Corning Lowell, Mass.) until confluent (membrane was fully covered with cells) and air-liquid interface (ALI) cultures were established by removing the apical media and culturing at 37° C./5% CO₂. Cells were cultured for more than 2 weeks at ALI before each experiment. Normal human bronchial epithelial (NHBE) cells (Scott Randell, University of North Carolina) were seeded at passage 2 on permeable membranes (12 mm Millicell, 0.4 μm pore size; Millipore Billerica, Mass.) and incubated (37° C., 5% CO₂, 95% RH) until confluent under liquid-covered culture conditions. Once confluent, the apical media was removed and ALI cultures were established. NHBE cells were cultured for 4 or more weeks at ALI prior to each experiment.

Prior to each experiment, the apical surface of each Transwell was washed 3 times with PBS. Where indicated, 10 μL of the specified TRP channel antagonist or agonist (see Table 2) was added to the apical surface of cells. As a control, cells that did not receive the channel antagonist or agonist were administered an equal volume of PBS (10 μL). When appropriate, cells were subsequently exposed to nebulized formulations (see Table 3) with a sedimentation chamber and Series 8900 nebulizers (Slater Labs). Immediately after exposure, the basolateral media (media on the bottom side of the Transwell or Millicell) was replaced with fresh media. Replicate wells were exposed to each formulation in each test. A second cell culture plate was exposed to the same formulations to quantify the delivery of total salt or calcium to cells. One hour after exposure, cells were infected with either 10 μL of Influenza A/Panama/2007/99 at a multiplicity of infection of 0.1-0.001 (0.1-0.001 virions per cell), 10 μL of rhinovirus (RV16) at a multiplicity of infection of 0.1-0.01 (0.1-0.01 virions per cell) or parainfluenza (hPIV3) at a multiplicity of infection of 3-0.3 (3-0.3 virions per cell). Four hours after aerosol treatment, the apical surfaces were washed to remove excess formulation and unattached virus. An additional 10 μL of the appropriate channel antagonist or agonist was added to the apical surface of the appropriate Transwells or Millicells and the plates were incubated overnight. Twenty four hours after viral infection, virus released onto the apical surface of infected cells was collected in culture media or PBS and the concentration of virus in the apical wash was quantified by a 50% Tissue Culture Infectious Dose assay (TCID₅₀). The TCID₅₀ assay is a standard endpoint dilution assay that is used to quantify how much of a given virus is present in a sample.

TABLE 2
TRP Channel Antagonists and Agonists

| | Antagonist/ | TRP channel | Working |
|---|---|---|---|
| Chemical Name | Agonist | target | Concentrations |
| Ruthenium Red | Antagonist | TRPV1-TRPV6, | 1 μM |
| | | TRPM6, TRPA1 | |
| SKF96365 | Antagonist | TRPV2, TRPC6, | −10 μM |
| | | TRPC7 | |
| RN1734 | Antagonist | TRPV4 | 1 μM-100 μM |
| 4αPDD | Agonist | TRPV4 | 0.01 μM-100 μM |
| GSK1016790A | Agonist | TRPV4 | 0.01 nm-1 μM |
| RN1747 | Agonist | TRPV4 | 1 nm-10 μM |
| Resiniferatoxin | Agonist | TRPV1 | 100 nm-100 μM |
| Menthol | Agonist | TRPM8, TRPV3 | 100 nm-100 μM |
| Carvacrol | Agonist | TRPA1, TRPV3 | 300 nm-100 μM |
| Icilin | Agonist | TRPA1, TRPM8 | 100 nm-100 μM |
| Flufenamic Acid | Agonist | TRPC6, TRPA1 | 100 nm-100 μM |

TABLE 3
Liquid Calcium-Sodium Formulations

| | CaCl₂ | CaCl₂ | NaCl | NaCl |
|---|---|---|---|---|
| Tonicity | (% w/v) | (M) | (% w/v) | (M) |
| 0.5X | 0.59 | 0.053 | 0.04 | 0.007 |
| 1X | 1.2 | 0.11 | 0.08 | 0.013 |
| (isotonic) | | | | |
| 2X | 2.4 | 0.21 | 0.16 | 0.027 |
| 4X | 4.7 | 0.43 | 0.31 | 0.053 |
| 8X | 9.4 | 0.85 | 0.62 | 0.11 |

### Example 1

### TRP Channel Antagonists Inhibit Calcium-Sodium Formulation-Mediated Reduction of Viral Infection

### A. TRP Channel Inhibition Reduces the Efficacy of a Calcium-Sodium Formulation Against Influenza.

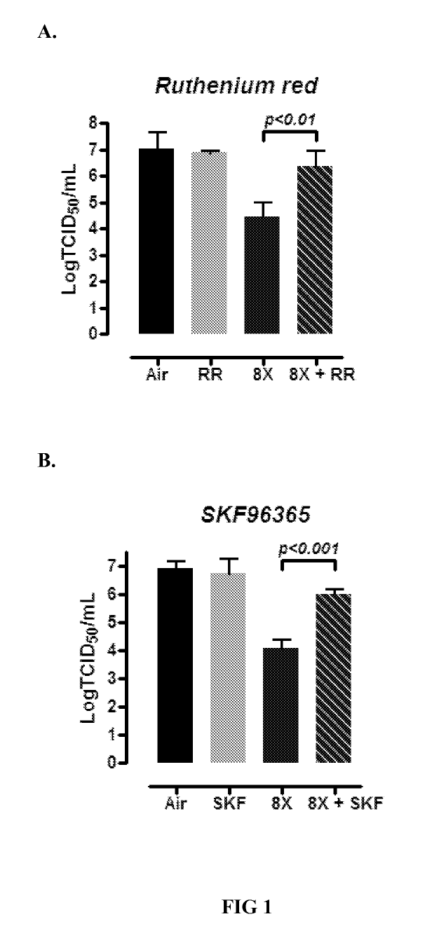

Calcium-sodium formulations inhibit viral infection (see, e.g., PCT/US10/28900 filed Mar. 26, 2010, incorporated herein by reference). To determine if calcium-sodium (Ca:Na) formulations act through TRP channels to reduce viral infection, ruthenium red (RR) and SKF96365, inhibitors of TRP channel activity (calcium uptake) (see Table 2), were employed. Calu-3 cells were pretreated with either 10 μL of RR (1 μM in PBS) or 10 μL of SKF96365 (10 μM in PBS) and subsequently exposed to an 8×Ca:Na formulation (see Table 3). Untreated cells or cells administered each treatment individually were used as controls. Treatment of cells with the 8×Ca:Na formulation significantly reduced influenza infection (140-fold) compared to the untreated control (FIG. 1; p<0.01 compared to untreated control; one-way ANOVA with Tukey's multiple comparison post-test). When the 8× treatment was administered to cells pretreated with either RR (FIG. 1A) or SKF96365 (FIG. 1B), the effect on viral infection was reduced to levels comparable to the untreated control cells. Each data point is the mean±standard deviation (SD) of triplicate wells and representative of two independent experiments with each antagonist. These results suggested that the inhibition of TRP channels by ruthenium red and SKF96365 reduced the efficacy of the 8×Ca:Na formulation against influenza.

### B. TRP Channel Inhibition Reduces the Efficacy of a Calcium-Sodium Formulation Against Parainfluenza and Rhinovirus.

Ruthenium red was used to determine if inhibition of TRP channel activity also reduced the previously demonstrated broad-spectrum anti-viral activity of Ca:Na formulations against other viruses like human parainfluenza virus and rhinovirus (see e.g., PCT/US10/28914 filed Mar. 26, 2010, incorporated herein by reference). Human parainfluenza virus is a single stranded RNA enveloped virus distinct from influenza. Parainfluenza viruses are 150-300 nm in diameter and are covered in hemagglutinin-neuraminidase (HN) spikes and fusion proteins. Unlike influenza virus, the genome is non-segmented and, following attachment of the virus to the target cell via HN tetramers, the virus is believed to fuse directly with the plasma membrane. Parainfluenza is associated with upper and lower respiratory tract disease and is frequently a cause of an influenza-like illness (ILI) and acute exacerbations (AEs) in respiratory infection. Human rhinovirus is a single-stranded RNA non-enveloped virus that causes the common cold. Among the smallest of viruses, rhinoviruses have a diameter of only about 30 nanometers. Human rhinoviruses are composed of a capsid made up of four viral proteins (VP1-VP4) that form an icosahedral structure. Rhinovirus preferentially infects the upper respiratory tract and enters respiratory epithelial cells through receptor-mediated (ICAM-1, LDL receptor family) endocytosis.

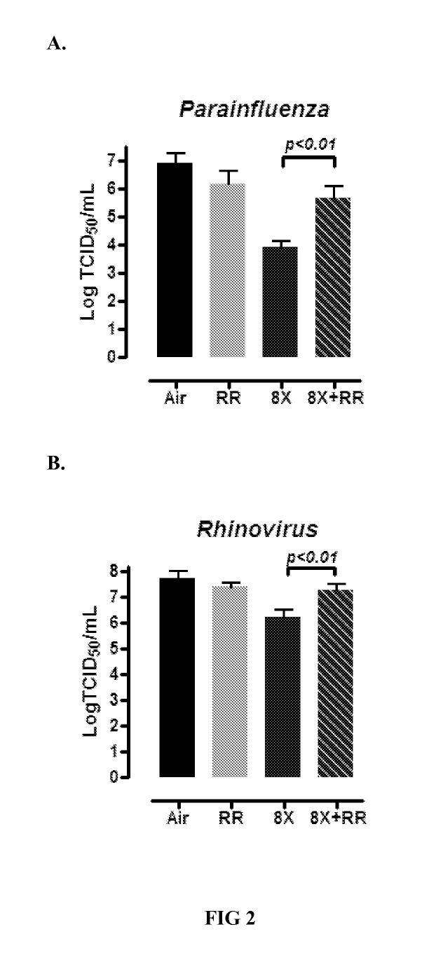

Ruthenium red and the 8×Ca:Na formulation were tested against parainfluenza virus and rhinovirus. Calu-3 cells were pretreated with ruthenium red (1 μM in PBS) and subsequently exposed to an 8×Ca:Na formulation. Untreated cells or cells administered each treatment individually were used as controls. Treatment of cells with the 8×Ca:Na formulation reduced parainfluenza (FIG. 2A) and rhinovirus (FIG. 2B) infection compared to the untreated control (FIG. 2; p<0.01 compared to untreated control; one-way ANOVA with Tukey's multiple comparison post-test). When the 8× treatment was administered to cells pretreated with ruthenium red, the efficacy of the 8× treatment was reduced (hatched bar in FIG. 2) and viral titers were comparable to those in the ruthenium red treated controls (grey bar in FIG. 2). Each data point is the mean±SD of triplicate wells.

### C. TRP Channel Inhibition Reduces the Efficacy of a Calcium-Sodium Formulation Against Influenza in Normal Human Bronchial Epithelial Cells.

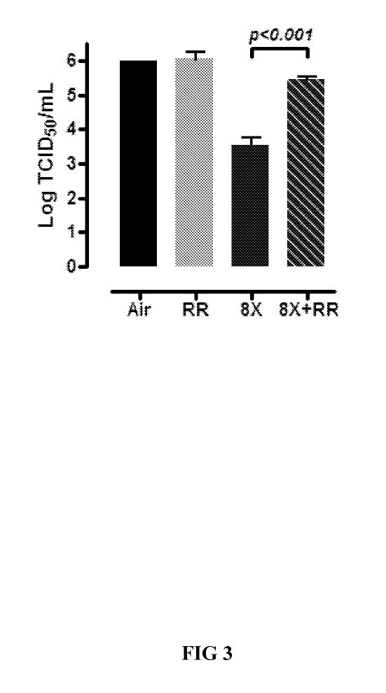

Normal human bronchial epithelial (NHBE) cells were also used to test the effect of TRP channel antagonism on Ca:Na formulation anti-viral activity. NHBE cell cultures are multicellular (ciliated epithelial cells, non-ciliated cells, and goblet cells) and are comprised of primary cells cultured from human lung tissue samples. Treatment of NHBE cells with Ca:Na formulations significantly reduced influenza infection and/or replication (see e.g., PCT/US10/28906 filed Mar. 26, 2010, incorporated herein by reference). NHBE cells were treated with ruthenium red (1 μM) immediately before treatment of cells with an 8×Ca:Na formulation. The 8×Ca:Na formulation significantly reduced influenza infection compared to untreated or ruthenium red treated cells in the absence of Ca:Na treatment (FIG. 3; p<0.001 compared to untreated control; one-way ANOVA with Tukey's multiple comparison post-test). However, treatment of cells with ruthenium red before treatment with the 8×Ca:Na formulation blocked the anti-viral action of the Ca:Na formulation; viral titers were similar to that of control cells (FIG. 3; p<0.001 compared to untreated control; one-way ANOVA with Tukey's multiple comparison post-test).

### D. TRP Channel Inhibition Reduces the Efficacy of a Dry Powder Calcium-Sodium Formulation Against Influenza.

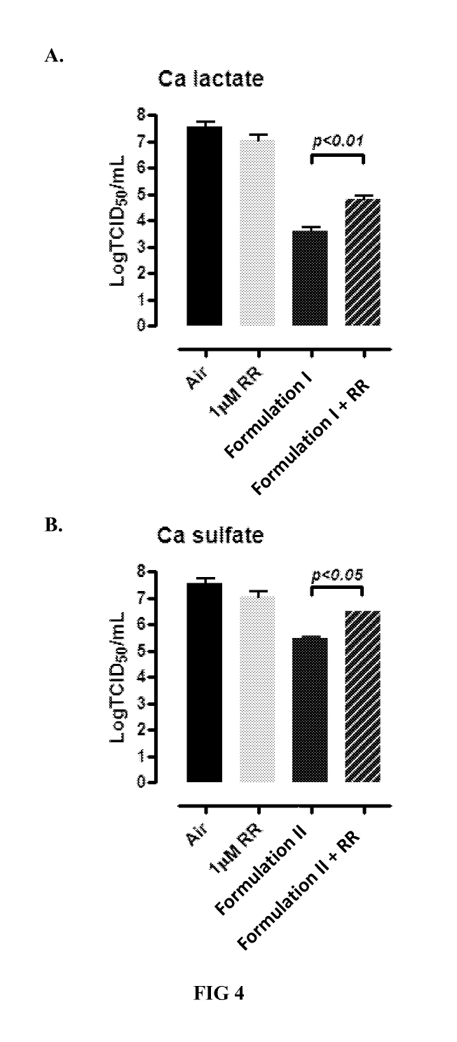

Dry powder Ca:Na formulations have also been shown to reduce viral infection (see e.g., PCT/US10/28900 and PCT/US10/28917 filed Mar. 26, 2010, incorporated herein by reference). To test the effect of TRP channel antagonists on the ability of dry powder Ca:Na formulations to inhibit viral replication, Calu-3 cells pre-treated with ruthenium red (1 μM) were subsequently treated with Formulation I (10% leucine, 58.6% calcium lactate, 31.4% sodium chloride, weight percent) or Formulation II (10% leucine, 39.6% calcium chloride, 50.4% sodium sulfate, weight percent). Three hours later, cells were washed to remove unattached virus, and again treated with ruthenium red. The delivered dose of Formulation I was 11.6 μg Ca/cm² alone and 12.1 μg Ca/cm² with ruthenium red. The delivered dose of Formulation II was 11.7 μg Ca/cm² alone and 11.8 μg Ca/cm² with ruthenium red. Compared to untreated or ruthenium red treated cells, treatment of cells with Formulation I reduced viral titer, and this reduction was attenuated by ruthenium red (FIG. 4A; p<0.01 compared to untreated control; one-way ANOVA with Tukey's multiple comparison post-test). Similarly, the reduction of influenza infection by treatment of cells with Formulation II was attenuated by ruthenium red (FIG. 4B; p<0.05 compared to untreated control; one-way ANOVA with Tukey's multiple comparison post-test). Each data point is the mean±SD of triplicate wells.

Altogether, these data suggested that Ca:Na formulations act through ruthenium red-sensitive TRP channels to reduce viral infection.

### Example 2

### Numerous TRP Channels are Expressed in Calu-3 and NHBE Cells

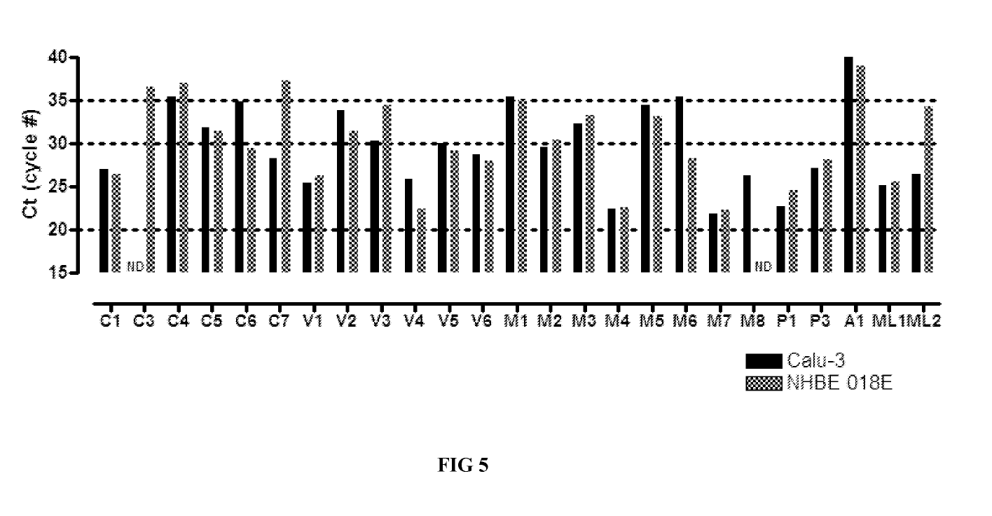

Twenty-eight different TRP channels have been identified in mammals. To determine which TRP channels were present and potentially being inhibited by ruthenium red in Calu-3 and NHBE cells, TRP channel gene expression was analyzed by quantitative PCR (qPCR). Quantitative PCR analysis was performed using total cellular RNA from Calu-3 and NHBE cells and oligonucleotides specific to each of the indicated channels (see FIG. 5). Two-step PCR reactions were performed with an initial reverse transcription reaction, followed by a second amplification reaction that contained SYBR green. The relative expression levels of each gene were determined using arbitrary cut-off points based on the expression of housekeeping genes (GAPDH) and negative control samples (no reverse transcription).

Of the 25 genes analyzed, 14 had cycle threshold (Ct) values of less than 30 in both cell types; thus, these 14 TRP channel genes would be predicted to be expressed in Calu-3 and NHBE cells. Channels with a Ct≦30 were: TRPC1, TRPC6 (NHBE only), TRPV1, TRPV4, TRPV5, TRPV6, TRPM2, TRPM4, TRPM6 (Calu-3 only), TRPM7, TRPM8 (Calu-3 only), TRPP1, TRPP3 and TRPML1 (FIG. 5). Six additional channels had Ct values between 30 and 35 and could possibly be expressed in each cell type. These 6 TRP channels with a Ct between 30 and 35 were TRPC5, TRPV2, TRPV3, TRPM3, TRPM5, and TRPML2 (FIG. 5). The remaining channels had Ct values greater than 35 and were likely not expressed at detectable levels. Of the channels that the qPCR analysis identified as being expressed in the cells, TRPV1, TRPV2, TRPV3, TRPV4, TRPV5, TRPV6, TRPM6 and TRPA1 have been described as being sensitive to ruthenium red. Thus, the qPCR analysis suggested that the aforementioned channels could be those through which Ca:Na formulations were exerting their anti-viral effect.

### Example 3

### The TRPV4 Channel is Involved in Ca:Na Formulation Reduction of Viral Infectivity

### A. TRPV4 Channel Inhibition Reduces Ca:Na Formulation Efficacy Against Influenza.

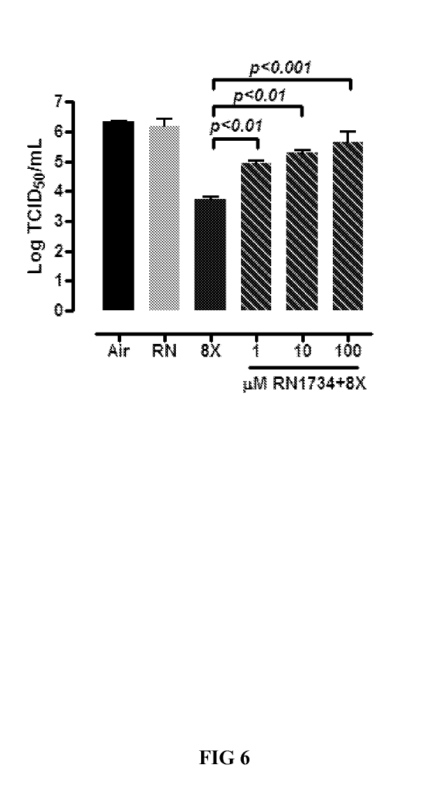

To test if one of the aforementioned TRP channels identified above, TRPV4, was involved in mediating Ca:Na formulation anti-viral activity, RN1734, a specific TRPV4 channel antagonist was tested for its ability to inhibit Ca:Na formulation reduction of viral infection. Calu-3 cells were either untreated or with increasing concentrations of RN1734 (1 μM, 10 μM and 100 μM). Cells were subsequently treated apically as described previously with an 8×Ca:Na formulation alone, or in wells treated with RN1734 at various concentrations. One hour later, cells were infected with influenza virus and 3 hours after that, washed to remove unattached virus, at which point the cells were treated again with RN1734. Twenty four hours after infection viral titers were determined from the apical surface of the cultures in a TCID₅₀ assay. RN1734 inhibited the 8×Ca:Na formulation-mediated reduction of influenza infection in a dose dependent manner (FIG. 6; p<0.01 (1 μM, 10 μM RN1734), p<0.001 (100 μM RN1734) compared to untreated control; one-way ANOVA with Tukey's multiple comparison post-test). Each bar depicts the mean±SD for each condition and data are pooled from two independent studies, each performed in duplicate. These data indicated that pretreatment of cells with a TRPV4 channel inhibitor blocked the anti-viral action of Ca:Na formulations and suggested that TRPV4 is one TRP channel through which Ca:Na formulations exert their effects.

### B. TRPV4 Activation Reduces Viral Infection.

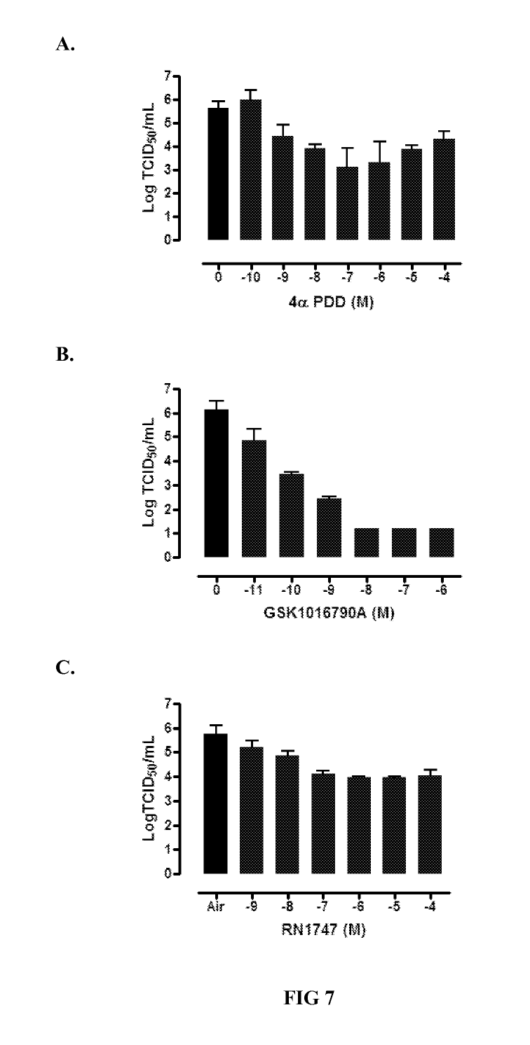

Given that Ca:Na formulations appeared to act through TRPV4 channels, activation of TRPV4 with specific agonists should mimic the anti-viral effects of Ca:Na formulations. TRPV4 agonists 4α-Phorbol 12,13-didecanoate (4αPDD) (Sigma-Aldrich, St. Louis, Mo.), GSK1016790A (Sigma-Aldrich, St. Louis, Mo.) and RN1747 (Menai Organics, Gwynedd, UK) were employed in these experiments. The apical surface of Calu-3 cells was treated with 10 μL of each TRPV4 agonist diluted to the indicated concentrations in PBS (see FIG. 7). One hour later, the cells were infected with Influenza A (H3N2). Three hours after infection, the apical surface of the Transwells was washed with PBS to remove unattached virus and the same concentrations of 4αPDD, GSK1016790A and RN1747 were added to the apical surface of the appropriate wells. Twenty-four hours after infection, virus released onto the apical surface of infected cells was collected in culture media or PBS, and the concentration of virus in the apical wash was quantified in a TCID₅₀ assay. The mean±SD of duplicate or triplicate wells is shown for each condition. All the TRPV4 agonists reduced influenza infection independent of the presence of Ca:Na formulations with GSK10168790, an extremely potent TRPV4 agonist (Thorneole et al., J Pharm Exp Ther 326:432-442, 2008), demonstrating the highest level of anti-viral efficacy in a dose-dependent manner. Overall, the data implicated TRPV4 as potentially mediating the anti-viral effect of the Ca:Na formulations.

### C. TPV4 Activation Reduces Parainfluenza and Rhinovirus Infection.

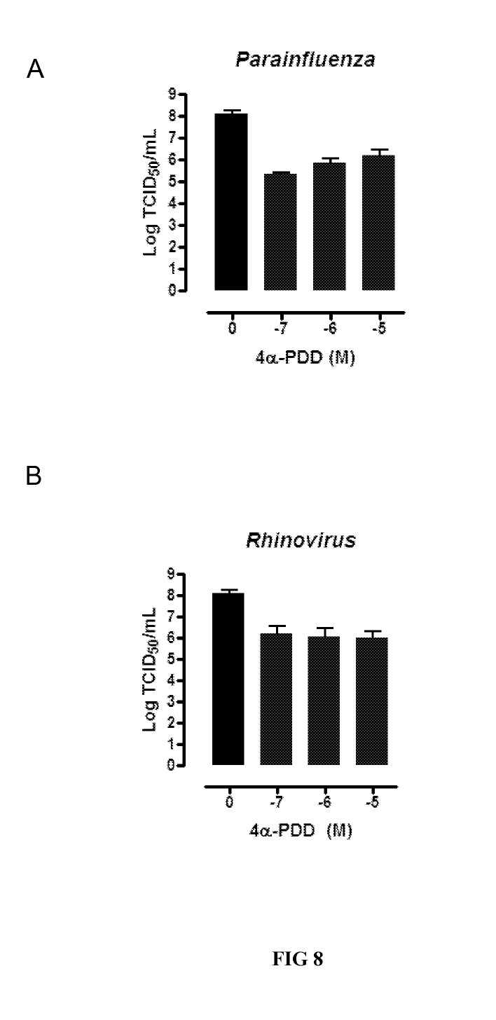

To determine if TRPV4 activation reduced infection of viruses other than influenza, TRPV4 agonists were also tested against parainfluenza and rhinovirus. Experiments were performed as described above for influenza virus. Briefly, Calu-3 cells were pretreated with different doses of 4αPDD (0.1 μM to 10 μM) in 10 μL PBS or with PBS alone. One hour later cells were infected with either parainfluenza (hPIV3) or rhinovirus (RV16). Cells were then washed 3 hours after infection to remove unattached virus and 4αPDD reapplied at the appropriate concentrations. Twenty-four hours after infection, viral titers were determined by a TCID₅₀ assay. The average ±SD of duplicate or triplicate wells is shown for each virus. 4αPDD was able to reduce both parainfluenza (FIG. 8A) and rhinovirus (FIG. 8B) infection. Thus, these results suggested that TRPV4 activation resulted in a broad-spectrum anti-viral activity.

### D. TRPV4 Activation Inhibits Influenza Infection of NHBE Cells.

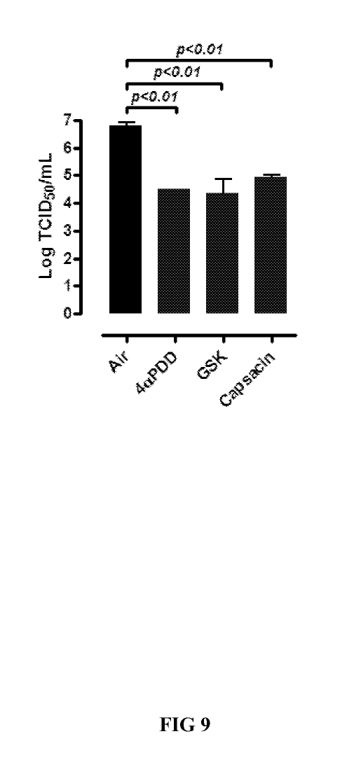

The anti-viral effect of TRPV4 activation was further tested in primary cells cultured from human lung tissue samples. NHBE cells were pre-treated with 4αPDD or GSK1016790A diluted in 10 μL of PBS to 1 μM one hour prior to infection with influenza. Cells were washed with PBS 3 hours after infection to remove unattached virus and agonists reapplied. After 24 hours, the apical surface of cells was rinsed and influenza viral titer assayed by TCID₅₀. The mean±SD of duplicate wells is shown for each condition. As seen in Calu-3 cells, treatment of NHBE cells with TRPV4 agonists reduced influenza replication compared to untreated (PBS control) cells (FIG. 9; p<0.01 compared to untreated control; one-way ANOVA with Tukey's multiple comparison post-test).

### Example 4

### Activation of Other TRP Channels Reduces Influenza Infection

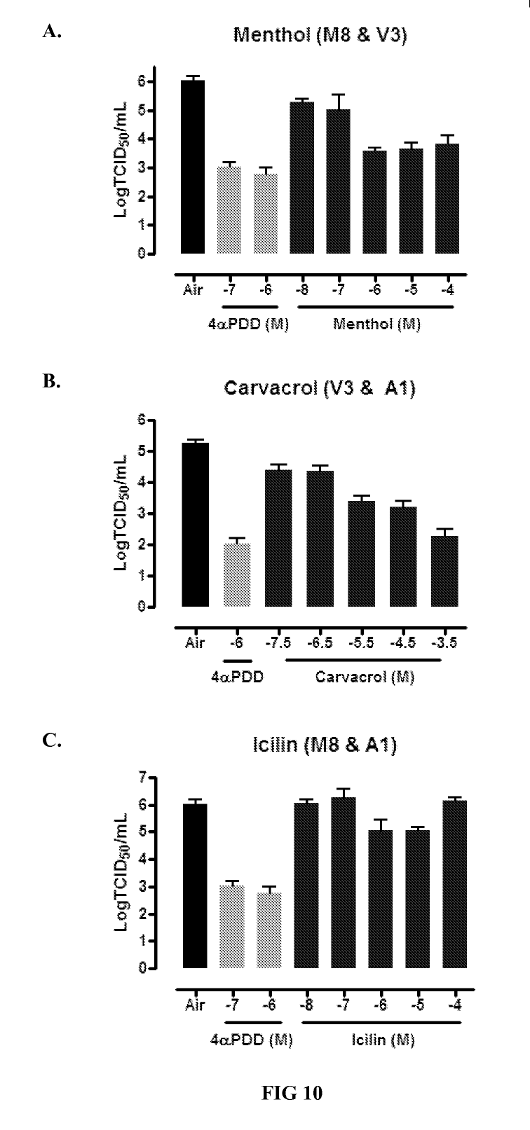

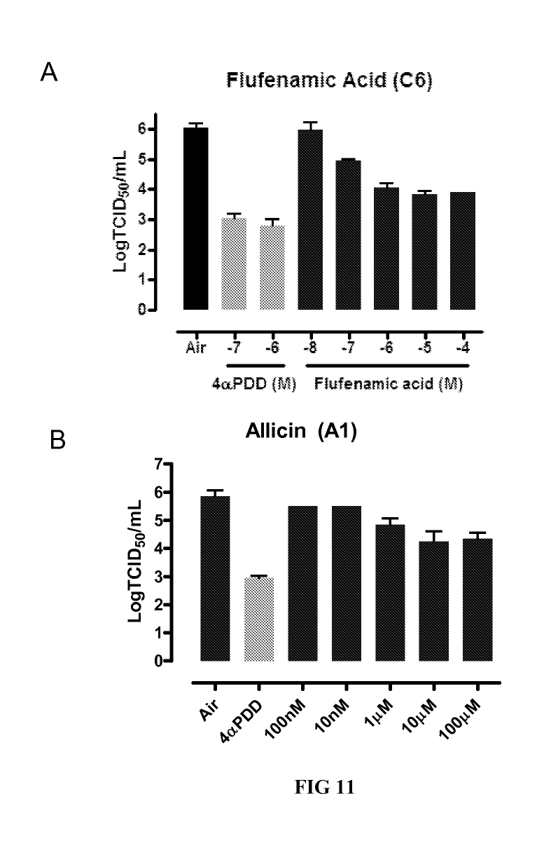

The activation of other TRP channels identified by qPCR analysis and those known to be sensitive to ruthenium red treatment were also tested for anti-viral activity using specific TRP channel agonists. Specifically, Calu-3 cells were pre-treated with the indicated concentrations of menthol (TRPM8 and TRPV3 agonist) (FIG. 10A), carvacrol (TRPV3 and TRPA1 agonist) (FIG. 10B), icilin (TRPM8 and TRPA1 agonist) (FIG. 10C), flufenamic acid (TRPC6 and TRPA1 agonist) (FIG. 11A) or allicin (TRPA1 agonist) (FIG. 11B) in 10 μL of PBS. In some studies, cells were also treated with 4αPDD (10 μL PBS) to assess the anti-viral activity of the TRP channel agonists relative to that resulting from TRPV4 activation. As described above, cells were infected with influenza virus 1 hour after agonist pre-treatment, washed 3 hours later with PBS, agonist reapplied at the appropriate concentrations and viral titer assayed (TCID₅₀) 24 hours after infection. The mean±SD of replicate wells is shown for each condition. Menthol (FIG. 10A), carvacrol (FIG. 10B), flufenamic acid (FIG. 11A) and allicin (FIG. 11B) all reduced influenza titer in a dose responsive manner, although none of these agonists were as effective as the TRPV4 agonist 4αPDD. Application of icilin resulted in a small, but measurable reduction of influenza titer (FIG. 10C). The level of reduction of influenza infection by the various TRP channel agonists tested is quantified and summarized in Table 4.

TABLE 4
Reduction of Influenza Infection

| | | Active | Max reduction in |
|---|---|---|---|
| Agonist | Target | concentrations | titer (Log10) |
| 4aPDD | TRPV4 | −9 to −5M | 2.9 |
| GSK1016790A | TRPV4 | −11 to −6M | 5.0 |
| RN1747 | TRPV4 | −8 to −4M | 2.3 |
| Capsacin | TRPV1 | −8 to −4M | 2.2 |
| Resiniferatoxin | TRPV1 | −6 to −4M | 3.3 |
| Allicin | TRPA1 | −6 to −4M | 1.6 |
| Icillin | TRPA1 | −6 to −4M | 1 |
| Carvacrol | TRPA1 | −6.5 to −4M | 3 |
| | TRPM8 | | |
| Menthol | TRPV3 | −7 to −4M | 2.5 |
| | TRPM8 | | |
| Flufenamic acid | TRPC6 | −6 to −4M | 2.5 |

In all, the data suggested that several TRP channels were involved in the observed anti-viral activity of Ca:Na formulations.

While this invention has been particularly shown and described with references to preferred embodiments thereof, it will be understood by those skilled in the art that various changes in form and details may be made therein without departing from the scope of the invention encompassed by the appended claims.

The teachings of all patent documents cited herein are incorporated herein by reference.

What is claimed is:

1. A method of treating or preventing a respiratory infection, comprising administering to an individual in need thereof an effective amount of an agonist of a TRP channel selected from the group consisting of TRPV2, TRPV3, TRPV4, TRPC6, TRPM6, TRPA1, and combinations thereof.

2. The method of claim 1, wherein said respiratory infection is a bacterial infection.

3. The method of claim 2, wherein said bacterial infection comprises infection by a pathogen selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp., Yersinia enterocolitica, Mycobacterium tuberculosis, Bordetella pertussis, Bordetella bronchiseptica, Brucella spp., Brucella abortus, Brucella melitensis, Brucella suis, Chlamydophila psittaci, Clostridium tetani, Streptococcus pyogenes, Corynebacterium diphtheriae, Neisseria meningitides, Enterococcus faecalis, Francisella tularensis, Bacillus anthracis, Helicobacter pylori, Leptospira spp, Leptospira interrogans, Listeria monocytogenes, Rickettsia rickettsii, Salmonella spp. Shigella sonnei, Vibrio cholerae, and Yersinia pestis.

4. The method of claim 1, wherein said respiratory infection is a viral infection.

5. The method of claim 4, wherein said viral infection comprises infection by a pathogen selected from the group consisting of influenza virus, rhinovirus, parainfluenza virus, respiratory syncytial virus (RSV), metapneumovirus, adenovirus, herpes simplex virus, cytomegalovirus (CMV), coronavirus, hantavirus, coxsackievirus, rhinovirus, enterovirus, human bocavirus (HBoV).

6. The method of claim 1, wherein said TRP channel agonist is administered as an aerosol to the respiratory tract of said individual.

7. The method of claim 1, further comprising administering one or more co-therapeutic agents selected from the group consisting of mucoactive or mucolytic agents, surfactants, cough suppressants, expectorants, steroids, bronchodilators, antihistamines, anti-inflammatory avents, antibiotics, and antiviral agents.

8. The method of claim 1, wherein an agonist of TRPV4 is administered.

9. A method of treating a respiratory infection, comprising administering to an individual having a respiratory infection an effective amount of a TRP channel agonist selected from the group consisting of Allyl isothiocyanate (AITC), Benyzl isothiocyanate (BITC), Phenyl isothiocyanate, Isopropyl isothiocyanate, methyl isothiocyanate, diallyl disulfide, acrolein (2-propenal), disulfuram (Antabuse®), farnesyl thiosalicylic acid (FTS), farnesyl thioacetic acid (FTA), chlodantoin (Sporostacin®, topical fungicidal), (15-d-PGJ2), 5,8,11,14 eicosatetraynoic acid (ETYA), dibenzoazepine, mefenamic acid, fluribiprofen, keoprofen, diclofenac, indomethacin, SC alkyne (SCA), pentenal, mustard oil alkyne (MOA), iodoacetamine, iodoacetamide alkyne, (2-aminoethyl) methanethiosulphonate (MTSEA), 4-hydroxy-2-noneal (HNE), 4-hydroxy xexenal (HHE), 2-chlorobenzalmalononitrile, N-chloro tosylamide (chloramine-T), formaldehyde, isoflurane, isovelleral, hydrogen peroxide, URB597, thiosulfinate, Allicin (a specific thiosulfinate), flufenamic acid, niflumic acid, carvacrol, eugenol, menthol, gingerol, icilin, methyl salicylate, arachidonic acid, cinnemaldehyde, super sinnemaldehyde, tetrahydrocannabinol (THC or Δ⁹-THC), cannabidiol (CBD), cannabichromene (CBC), cannabigerol (CBG), THC acid (THC-A), CBD acid (CBD-A), Compound 1 (AMG5445), 4-methyl-N-[2,2,2-trichloro-1-(4-chlorophenylsulfanyl)ethyl]benzamide, N-[2,2,2-trichloro-1-(4-chlorophenylsulfanyl)ethyl]acetamid, AMG9090, AMG5445, 1-oleoyl-2-acetyl-sn-glycerol (OAG), carbachol, diacylglycerol (DAG), 1,2-Didecanoylglycerol, flufenamate/flufenamic acid, niflumate/niflumic acid, hyperforin, 2-aminoethoxydiphenyl borate (2-APB), diphenylborinic anhydride (DPBA), delta-9-tetrahydrocannabinol (Δ⁹-THC or THC), cannabiniol (CBN), 2-APB, O-1821, 11-hydroxy-Δ⁹-tetrahydrocannabinol, nabilone, CP55940, HU-210, HU-211/dexanabinol, HU-331, HU-308, JWH-015, WIN55,212-2, 2-Arachidonoylglycerol (2-AG), Arvil, PEA, AM404, O-1918, JWH-133, incensole, incensole acetate, menthol, eugenol, dihydrocarveol, carveol, thymol, vanillin, ethyl vanillin, cinnemaldehyde, 2 aminoethoxydiphenyl borate (2-APB), diphenylamine (DPA), diphenylborinic anhydride (DPBA), camphor, (+)-borneol, (−)-isopinocampheol, (−)-fenchone, (−)-trans-pinocarveol, isoborneol, (+)-camphorquinone, (−)-α-thujone, α-pinene oxide, 1,8-cineole/eucalyptol, 6-tert-butyl-m-cresol, carvacrol, p-sylenol, kreosol, propofol, p-cymene, (−)-isoppulegol, (−)-carvone, (+)-dihydrocarvone, (−)-menthone, (+)-linalool, geraniol, 1-isopropyl-4-methyl-bicyclo[3.1.0]hexan-4-ol, 4αPDD, GSK1016790A, 5′6′Epoxyeicosatrienoic 8′9′Epoxyeicosatrienoic (8′9′-EET), APP44-1, RN1747, Formulation Ib WO200602909, Formulation IIb WO200602909, Formulation IIc WO200602929, Formulation IId WO200602929, Formulation IIIb WO200602929, Formulation IIIc WO200602929, arachidonic acid (AA), 12-O-Tetradecanoylphorbol-13-acetate (TPA)/phorbol 12-myristate 13-acetate (PMA), bisandrographalide (BAA), incensole, incensole acetate, Compound IX WO2010015965, Compound X WO2010015965, Compound XI WO2010015965, Compound XII WO2010015965, WO2009004071, WO2006038070, WO2008065666, Formula VII WO2010015965, Formula IV WO2010015965, dibenzoazepine, dibenzooxazepine, Formula I WO2009071631, N-{(1S)-1-[({(4R)-1-[(4-chlorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(4-fluorophenyl)sulfonyl]-3-oxohexahydro-1′-1-azepin-4-yl}amino)carbonyl]-3-methylbutyl}-1-benzothiophen-2-carboxamide, N-{(1S)-1-[({(4R)-1-[(2-cyanophenyl)sulfonyl]-3-oxohexahydro-1H-azepin-4-yl}amino)carbonyl]-3-methylbutyl}-1-methyl-1H-indole-2-carboxamide, and N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide.

10. The method of claim 9, wherein said respiratory infection is a bacterial infection.

11. The method of claim 10, wherein said bacterial infection comprises infection by a pathogen selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, catarrhalis, Chlamydophila pneumoniae, Mycoplasma pneumoniae, Legionella pneumophila, Enterobacter spp., Acinetobacter spp. Acinetobacter baumannii, methicillin-resistant Staphylococcus aureus, Stenotrophomonas maltophilia, Burkholderia spp., Yersinia enterocolitica, Mycobacterium tuberculosis, Bordetella pertussis, Bordetella bronchiseptica, Brucella spp., Brucella abortus, Brucella melitensis, Brucella suis, Chlamydophila psittaci, Clostridium tetani, Streptococcus pyogenes, Corynebacterium diphtheriae, Neisseria meningitides, Enterococcus faecalis, Francisella tularensis, Bacillus anthracis, Helicobacter pylori, Leptospira spp., Leptospira interrogans, Listeria monocytogenes, Rickettsia rickettsii, Salmonella spp., Shigella sonnei, Vibrio cholerae, and Yersinia pestis.

12. The method of claim 9, wherein said respiratory infection is a viral infection.

13. The method of claim 12, wherein said viral infection comprises infection by a pathogen selected from the group consisting of influenza virus, rhinovirus, parainfluenza virus, respiratory syncytial virus (RSV), metapneumovirus, adenovirus, herpes simplex virus, cytomegalovirus (CMV), coronavirus, hantavirus, coxsackievirus, rhinovirus, enterovirus, human bocavirus (HBoV).

14. The method of claim 9, wherein said TRP channel agonist is administered as an aerosol to the respiratory tract of said individual.

15. The method of claim 9, further comprising administering one or more co-therapeutic agents selected from the group consisting of mucoactive or mucolytic agents, surfactants, cough suppressants, expectorants, steroids, bronchodilators, antihistamines, anti-inflammatory agents, antibiotics, and antiviral agents.

16. A method of treating a respiratory infection, comprising administering to an individual having a respiratory infection an effective amount of an agonist of TRPV4, wherein the agonist is selected from the group consisting of 4αPDD, GSK1016790A, and RN1747.

17.-18. (canceled)
